(19) United States
(12) Patent Application Publication
(10) Pub. No.: US 2003/0207300 A1
(43) Pub. Date: Nov. 6, 2003

(54) Multiplex analytical platform using molecular tags

(76) Inventors: Tracy J. Matray, Campbell, CA (US); Sharat S. Singh, San Jose, CA (US); Stephen C. Macevicz, Cupertino, CA (US)

Correspondence Address: ACLARA BIOSCIENCES, INC., 1288 PEAR AVENUE, MOUNTAIN VIEW, CA, 94043, US

(21) Appl. No.: 10/338,729

(22) Filed: Jan. 7, 2003

Related U.S. Application Data
(63) Continuation-in-part of application No. 10/154,042, filed on May 21, 2002.
Application No. 10/154,042 is a continuation-in-part of application No. 09/698,846, filed on Oct. 27, 2000.
Application No. 09/698,846 is a continuation-in-part of application No. 09/602,586, filed on Jun. 21, 2000, now Pat. No. 6,514,700.
Application No. 09/602,586 is a continuation-in-part of application No. 09/561,579, filed on Apr. 28, 2000, now abandoned.

(51) Int. Cl. C12Q 1/68; C12P 19/34

(52) U.S. Cl. 435/6; 435/91.2

(57) ABSTRACT

Compositions and methods are disclosed for detecting multiple target analytes, particularly polynucleotide target analytes. In accordance with one aspect of the invention, a template-dependent extension reaction is performed to generate detection probes, such that each detection probe has (i) at least one molecular tag attached by a cleavable linkage and (ii) either a capture moiety or a cleavage-inducing moiety attached. The template-dependent extension reaction may be carried out directly on a polynucleotide analyte to generate molecular tags, wherein the polynucleotide analyte serves as a template in the template-dependent extension reaction, or it may be carried out indirectly on an oligonucleotide label that, in turn, is attached to a binding moiety specific for an analyte of interest. In either case, a plurality of molecular tags are generated, after which they are separated and identified to determine the presence or absence or the quantity of the target analytes in a sample.

This is a continuation-in-part of co-pending U.S. application Ser. No. 10/154,042 filed 21 May 2002, which is a continuation-in-part of co-pending U.S. application Ser. No. 09/698,846 filed 27 Oct. 2000, which is a continuation-in-part of co-pending U.S. application Ser. No. 09/602,586 filed 21 Jun. 2000, which is a continuation-in-part of co-pending U.S. application Ser. No. 09/561,579 filed 28 Apr. 2000. All of the above-referenced applications are hereby incorporated by reference.

### FIELD OF THE INVENTION

This invention relates to methods and compositions for detecting and/or measuring multiple analytes in a sample using a nucleic acid-based signal amplification system for simultaneous generation of multiple molecular tags.

### BACKGROUND OF THE INVENTION

The development of several powerful technologies for genome-wide and proteome-wide expression measurements has created an opportunity to study and understand the coordinated activities of large sets of, if not all, an organism's genes in response to a wide variety of conditions and stimuli, e.g. DeRisi et al, Science, 278: 680-686 (1997); Wodicka et al, Nature Biotechnology, 15: 1359-1367 (1997); Velculescu et al, Cell, 243-251 (1997); Brenner et al, Nature Biotechnology, 18: 630-634 (2000); McDonald et al, Disease Markers, 18: 99-105 (2002); Patterson, Bioinformatics, 18 (Suppl 2): S181 (2002). Studies using these technologies have shown that reduced subsets of genes appear to be co-regulated to perform particular functions and that subsets of expressed genes and proteins can be used to classify cells phenotypically, e.g. Shiffman and Porter, Current Opinion in Biotechnology, 11: 598-601 (2000); Afshari et al, Nature, 403: 503-511 (2000); Golub et al, Science, 286: 531-537 (1999); van't Veer et al, Nature, 415: 530-536 (2002); and the like.

An area of interest in drug development is the expression profiles of genes and proteins involved with the metabolism or toxic effects of xenobiotic compounds. Several studies have shown that sets of several tens of genes can serve as indicators of compound toxicity, e.g. Thomas et al, Molecular Pharmacology, 60: 1189-1194 (2001); Waring et al, Toxicology Letters, 120: 359-368 (2001); Longueville et al, Biochem. Pharmacology, 64: 137-149 (2002); and the like. Similarly, in the area of cancer diagnostics and prognosis, the differential expression of sets of a few tens of genes or proteins has been shown frequently have strong correlations with the progression and prognosis of a cancer.

Another area of interest in drug development is the expression and interation of different receptor types on the surface membranes of biological cells to form functional hetero-oligomeric receptors, e.g. George et al, Nature Reviews Drug Discovery, 1: 808-820 (2002). Monitoring such associations may require simultaneous measurement of several different events taking place on cell surface membranes of a population of cells. For example, if three interacting receptor components are present, then six homodimer and heterodimer combinations, or pairing events, are possible.

Accordingly, there is an interest in technologies that provide convenient and accurate measurements of multiple expressed genes in a single assay, either at the messenger RNA level or the protein level, or both. Current approaches to such measurements include multiplexed polymerase chain reaction (PCR), spotted and synthesized DNA microarrays, color-coded microbeads, and single-analyte assays, such as enzyme-linked immunosorbant assays (ELISAs) or Taqman-based PCR, used with robotics apparatus, e.g. Longueville et al (cited above); Elnifro et al, Clinical Microbiology Reviews, 13: 559-570 (2000); Chen et al, Genome Research, 10: 549-557 (2000); and the like. In regard to cell surface membrane receptor interactions, current approaches include co-immunoprecipitation and fluorescence or bioluminescence resonance energy transter methods, e.g. Angers et al, Proc. Natl. Acad., Sci., 97: 3684-3689 (2000); Kroeger et al, J. Biol. Chem., 276: 12736-12743 (2001).

Unfortunately, none of the above approaches provides a completely satisfactory solution for the desired measurements for several reasons including difficulty in automating, reagent usage, lack of convenience, lack of sensitivity, difficulty in obtaining consistent results, and so on, e.g. Elnifro et al (cited above); Hess et al, Trends in Biotechnology, 19: 463-468 (2001); King and Sinha; JAMA, 286: 2280-2288 (2001).

In view of the above, the availability of a convenient and cost effective technique for measuring the presence or absence or quantities of multiple analytes, such as gene expression products, in a single assay reaction would advance the art in many fields where such measurements are becoming increasingly important, including life science research, medical research and diagnostics, drug discovery, genetic identification, animal and plant science, and the like.

### SUMMARY OF THE INVENTION

The present invention is directed to methods and compositions for generating a plurality of distinct molecular tags indicative of the presence or absence of one or more analytes or the amounts of one or more analytes in a sample. In one aspect, the invention is directed to methods and compositions for determining the presence or the amounts of a plurality of target nucleic acid sequences in a sample by generating unique and readily measured molecular tags whenever a target nucleic acid sequence is present. The molecular tags of the plurality differ from one another by one or more physical or optical characteristics so that after they are generated in a reaction they may be separated and identified based on such differences.

In accordance with the invention, a template-dependent extension reaction is performed to generate detection probes, such that each detection probe has (i) at least one molecular tag attached by a cleavable linkage and (ii) either a capture moiety or a cleavage-inducing moiety attached. The template-dependent extension reaction may be carried out directly on a polynucleotide analyte to generate molecular tags, wherein the polynucleotide analyte serves as a template in the template-dependent extension reaction, or it may be carried out indirectly on an oligonucleotide label that, in turn, is attached to a binding moiety specific for an analyte of interest. In either case, a plurality of molecular tags are generated, after which they are separated and identified to determine the presence or absence or the quantity of the target analytes in a sample.

In one aspect of the invention, a primer is annealed to each target sequence under reaction conditions that permit the primer to be extended in a template-dependent reaction to form a detection probe. Each target sequence may be responsible for the generation of multiple detection probes by multiple cycles of annealing, extension, and dissociation, which may take place either isothermally or through thermal cycling. In either case, the reaction is continued until a detectable amount of detection probe is generated.

In another aspect, the invention includes compositions comprising detection probes and intermediates for the synthesis of detection probes. In particular, intermediates of the invention include molecular tag-labeled nucleoside triphosphates and photosensitizer-labeled nucleoside triphosphates used to form detection probes in an extension reaction with a polymerase.

In another aspect, the present invention includes kits for performing the methods of the invention. In one embodiment, such kits comprise a mixture of primers specific for a plurality of target nucleic acid sequences, the primers having either a sensitizer attached or at least one molecular tag attached by a cleavable linkage. Such kits further comprise additional components of an extension reaction so that complete detection probes may be formed, such as a polymerase and molecular tag-labeled or photosensitizer-labeled nucleoside triphosphates, a ligase and sensitizer-labeled oligonucleotides, or the like. Such kits may further include appropriate buffers for carrying out the extension reactions, capture agents to isolate detection probes, cleavage agents, agents to activate sensitizers, and the like. In another embodiment, kits may include binding moieties, such as antibodies, attached to oligonucleotide labels such that the oligonucleotide labels serve as templates in the method of the invention.

The present invention provides a detection and signal generation means with several advantages for multiplexed measurements of target analytes, including but not limited to (1) the detection and/or measurement of molecular tags that are separated from the assay mixture provide greatly reduced background and a significant gain in sensitivity; (2) the use of tags that are specially designed for ease of separation thereby providing convenient multiplexing capability; and (3) in many embodiments, providing greater sensitivity by forming a detection probe in situ by a single-nucleotide polymerase extention reaction.

### BRIEF DESCRIPTION OF THE DRAWINGS

FIG. 1A illustrates an embodiment of the invention wherein detection probes are generated in a reaction in which a polymerase extends a molecular tag-labeled primer by a single nucleotide having a photosensitizer attached.

FIG. 1B illustrates an embodiment of the invention wherein detection probes are generated in a reaction in which a polymerase extends a molecular tag-labeled primer by a single nucleotide having a biotin attached to form a detection probe which, in turn, is captured by an streptavidinated sensitizer bead.

FIG. 1C illustrates an embodiment of the invention wherein detection probes are generated by carrying out a ligation reaction that in the presence of a target nucleotide sequence couples a molecular tag-labeled primer with a photosensitizer-labeled oligonucleotide.

FIG. 1D illustrates an embodiment of the invention wherein detection probes are generated by carrying out a ligation reaction that in the presence of a target nucleotide sequence couples a molecular tag-labeled primer with a biotin-labeled oligonucleotide which, in turn, is captured by an streptavidinated sensitizer bead.

FIG. 1E illustrates how the distance between an attached molecular tag and a photosensitizer decreases upon melting or exchange from a template by the formation of a random coil configuration.

FIG. 1F illustrates an embodiment of the invention wherein detection probes are generated by carrying out a PCR with a molecular tag-labeled primer and a biotinylated primer, after which biotinylated polynucleotides are captured on streptavidinated sensitizer beads for release of the molecular tags.

FIG. 1G illustrates an embodiment of the invention wherein detection probes are generated by an RNA polymerase acting on an antibody-oligonucleotide conjugate having a promoter site for the polymerase and a coded sequence. Detection probes are the oligoribonucleotide reaction products of the RNA polymerase having a biotinylated base and a molecular tag-labeled base.

FIG. 1H illustrates an embodiment similar to that of FIG. 1G wherein two antibodies bind to a target analyte so that a double stranded oligonucleotide label forms that contains an RNA polymerase recognition site and coded sequence for generating detection probes.

FIG. 1I is a synthetic scheme for producing a protected phosphoramidite for introducing a cleavable linker at the 5′ end of an oligonucleotide. This permits molecular tags to be conveniently attached using a conventional automated DNA synthesizer.

FIG. 2 illustrates one exemplary synthetic approach starting with commercially available 6-carboxy fluorescein, where the phenolic hydroxyl groups are protected using an anhydride. Upon standard extractive workup, a 95% yield of product is obtained. This material is phosphitylated to generate the phosphoramidite monomer.

FIG. 3 illustrates the use of a symmetrical bis-amino alcohol linker as the amino alcohol with the second amine then coupled with a multitude of carboxylic acid derivatives.

FIG. 4 shows the structure of several benzoic acid derivatives that can serve as mobility modifiers.

FIG. 5 illustrates the use of an alternative strategy that uses 5-aminofluorescein as starting material and the same series of steps to convert it to its protected phosphoramidite monomer.

FIG. 6 illustrates several amino alcohols and diacid dichlorides that can be assembled into mobility modifiers in the synthesis of molecular tags.

FIGS. 7A-F illustrate oxidation-labile linkages and their respective cleavage reactions mediated by singlet oxygen.

FIGS. 8A-B illustrate the general methodology for conjugation of an e-tag moiety to an antibody to form an e-tag probe, and the reaction of the resulting probe with singlet oxygen to produce a sulfinic acid moiety as the released molecular tag.

FIGS. 9A-J show the structures of e-tag moieties that have been designed and synthesized.

FIGS. 10A-I illustrate the chemistries of synthesis of the e-tag moieties illustrated in FIG. 9.

FIGS. 11A-D illustrate exemplary photosensitizer molecules that may be attached to nucleoside triphosphates of the invention.

### DEFINITIONS

“Analyte” means a substance, compound, or component in a sample whose presence or absence is to be detected or whose quantity is to be measured. Analytes include but are not limited to peptides, proteins, polynucleotides, polypeptides, oligonucleotides, organic molecules, haptens, epitopes, parts of biological cells, posttranslational modifications of proteins, receptors, complex sugars, vitamins, hormones, and the like. There may be more than one analyte associated with a single molecular entity, e.g. different phosphorylation sites on the same protein.

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained.

“Antibody binding composition” means a molecule or a complex of molecules that comprise one or more antibodies and derives its binding specificity from an antibody. Antibody binding compositions include, but are not limited to, antibody pairs in which a first antibody binds specifically to a target molecule and a second antibody binds specifically to a constant region of the first antibody; a biotinylated antibody that binds specifically to a target molecule and streptavidin derivatized with moieties such as molecular tags or photosensitizers; antibodies specific for a target molecule and conjugated to a polymer, such as dextran, which, in turn, is derivatized with moieties such as molecular tags or photosensitizers; antibodies specific for a target molecule and conjugated to a bead, or microbead, or other solid phase support, which, in turn, is derivatized with moieties such as molecular tags or photosensitizers, or polymers containing the latter.

“Capillary-sized” in reference to a separation column means a capillary tube or channel in a plate or microfluidics device, where the diameter or largest dimension of the separation column is between about 25-500 microns, allowing efficient heat dissipation throughout the separation medium, with consequently low thermal convection within the medium.

“Chromatography” or “chromatographic separation” as used herein means or refers to a method of analysis in which the flow of a mobile phase, usually a liquid, containing a mixture of compounds, e.g. molecular tags, promotes the separation of such compounds based on one or more physical or chemical properties by a differential distribution between the mobile phase and a stationary phase, usually a solid. The one or more physical characteristics that form the basis for chromatographic separation of analytes, such as molecular tags, include but are not limited to molecular weight, shape, solubility, pKa, hydrophobicity, charge, polarity, and the like. In one aspect, as used herein, “high pressure (or performance) liquid chromatography” (“HPLC”) refers to a liquid phase chromatographic separation that (i) employs a rigid cylindrical separation column having a length of up to 300 mm and an inside diameter of up to 5 mm, (ii) has a solid phase comprising rigid spherical particles (e.g. silica, alumina, or the like) having the same diameter of up to 5 μm packed into the separation column, (iii) takes place at a temperature in the range of from 35° C. to 80° C. and at column pressure up to 150 bars, and (iv) employs a flow rate in the range of from 1 μL/min to 4 μL/min. Preferably, solid phase particles for use in HPLC are further characterized in (i) having a narrow size distribution about the mean particle diameter, with substantially all particle diameters being within 10% of the mean, (ii) having the same pore size in the range of from 70 to 300 angstroms, (iii) having a surface area in the range of from 50 to 250 m²/g, and (iv) having a bonding phase density (i.e. the number of retention ligands per unit area) in the range of from 1 to 5 per nm². Exemplary reversed phase chromatography media for separating molecular tags include particles, e.g. silica or alumina, having bonded to their surfaces retention ligands, such as phenyl groups, cyano groups, or aliphatic groups selected from the group including C₈ through C₁₈. Chromatography in reference to the invention includes “capillary electrochromatography” (“CEC”), and related techniques. CEC is a liquid phase chromatographic technique in which fluid is driven by electroosmotic flow through a capillary-sized column, e.g. with inside diameters in the range of from 30 to 100 μm. CEC is disclosed in Svec, Adv. Biochem. Eng. Biotechnol. 76: -147 (2002); Vanhoenacker et al, Electrophoresis, 22: 4064-4103 (2001); and like references. CEC column may use the same solid phase materials as used in conventional reverse phase HPLC and additionally may use so-called “monolithic” non-particular packings. In some forms of CEC, pressure as well as electroosmosis drives an analyte-containing solvent through a column.

The term “isothermal” in reference to assay conditions means a uniform or constant temperature at which the cleavage of the binding compound in accordance with the present invention is carried out. The temperature is chosen so that the duplex formed by hybridizing the probes to a polynucleotide with a target polynucleotide sequence is in equilibrium with the free or unhybridized probes and free or unhybridized target polynucleotide sequence, a condition that is otherwise referred to herein as “reversibly hybridizing” the probe with a polynucleotide. Normally, at least 1%, preferably 20 to 80%, usually less than 95% of the polynucleotide is hybridized to the probe under the isothermal conditions. Accordingly, under isothermal conditions there are molecules of polynucleotide that are hybridized with the probes, or portions thereof, and are in dynamic equilibrium with molecules that are not hybridized with the probes. Some fluctuation of the temperature may occur and still achieve the benefits of the present invention. The fluctuation generally is not necessary for carrying out the methods of the present invention and usually offer no substantial improvement. Accordingly, the term “isothermal” includes the use of a fluctuating temperature, particularly random or uncontrolled fluctuations in temperature, but specifically excludes the type of fluctuation in temperature referred to as thermal cycling, which is employed in some known amplification procedures, e.g., polymerase chain reaction.

“Kit” as used herein refers to any delivery system for delivering materials. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Nucleobase” means a nitrogen-containing heterocyclic moiety capable of forming Watson-Crick type hydrogen bonds with a complementary nucleobase or nucleobase analog, e.g. a purine, a 7-deazapurine, or a pyrimidine. Typical nucleobases are the naturally occurring nucleobases adenine, guanine, cytosine, uracil, thymine, and analogs of naturally occurring nucleobases, e.g. 7-deazaadenine, 7-deaza azaadenine, 7-deazaguanine, 7-deaza azaguanine, inosine, nebularine, nitropyrrole, nitroindole, 2-amino-purine, 2,6-diaminopurine, hypoxanthine, pseudouridine, pseudocytidine, pseudoisocytidine, 5-propynylcytidine, isocytidine, isoguanine, 2-thiopyrimidine, 6-thioguanine, 4-thiothymine, 4-thiouracil, O6-methylguanine, N6-methyl-adenine, O4-methylthyrine, 5,6-dihydrothymine, 5,6-dibydrouracil, 4-methylindole, and ethenoadenine, e.g. Fasman, Practical Handbook of Biochemistry and Molecular Biology, pp. 385-394, CRC Press, Boca Raton, Fla. (1989).

“Nucleoside” means a compound comprising a nucleobase linked to a C-1′ carbon of a ribose sugar or analog thereof. The ribose or analog may be substituted or unsubstituted. Substituted ribose sugars include, but are not limited to, those riboses in which one or more of the carbon atoms, preferably the 3′-carbon atom, is substituted with one or more of the same or different substituents such as —R, —OR, —NRR or halogen (e.g., fluoro, chloro, bromo, or iodo), where each R group is independently —H, C1-C6 alkyl or C3-C14 aryl. Particularly preferred riboses are ribose, 2′-deoxyribose, 2′,3′-dideoxyribose, Y-haloribose (such as 3′-fluororibose or 3′-chlororibose) and 3′-alkylribose. Typically, when the nucleobase is A or G, the ribose sugar is attached to the N9-position of the nucleobase. When the nucleobase is C, T or U, the pentose sugar is attached to the N′-position of the nucleobase (Komberg and Baker, DNA Replication, 2 d Ed., Freeman, San Francisco, Calif., (1992)). Examples of ribose analogs include arabinose, 2′-O-methyl ribose, and locked nucleoside analogs (e.g., WO 99/14226), for example, although many other analogs are also known in the art.

“Nucleotide” means a phosphate ester of a nucleoside, either as an independent monomer or as a subunit within a polynucleotide. Nucleotide triphosphates are sometimes denoted as “NTP”, “dNTP” (2′-deoxypentose) or “ddNTP” (2′,3′-dideoxypentose) to particularly point out the structural features of the ribose sugar. “Nucleoside 5′-triphosphate” refers to a nucleotide with a triphosphate ester group at the 5′ position. The triphosphate ester group may include sulfur substitutions for one or more phosphate oxygen atoms, e.g. α-thionucleoside 5′-triphosphates.

“Oligonucleotide” as used herein means linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides of the invention comprise the four natural deoxynucleotides; however, they may also comprise ribonucleosides or non-natural nucleotide analogs. It is clear to those skilled in the art when oligonucleotides having natural or non-natural nucleotides may be employed in the invention. For example, where processing by an enzyme is called for, usually oligonucleotides consisting of natural nucleotides are required. Likewise, where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding. As used herein, “stable duplex” between complementary oligonucleotides or polynucleotides means that a significant fraction of such compounds are in duplex or double stranded form with one another as opposed to single stranded form. Preferably, such significant fraction is at least ten percent of the strand in lower concentration, and more preferably, thirty percent.

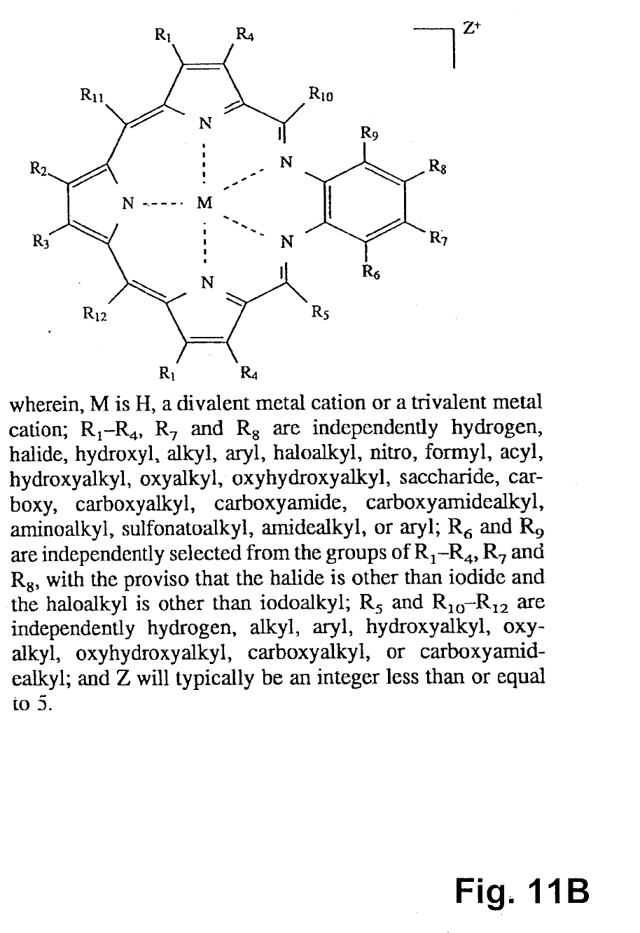

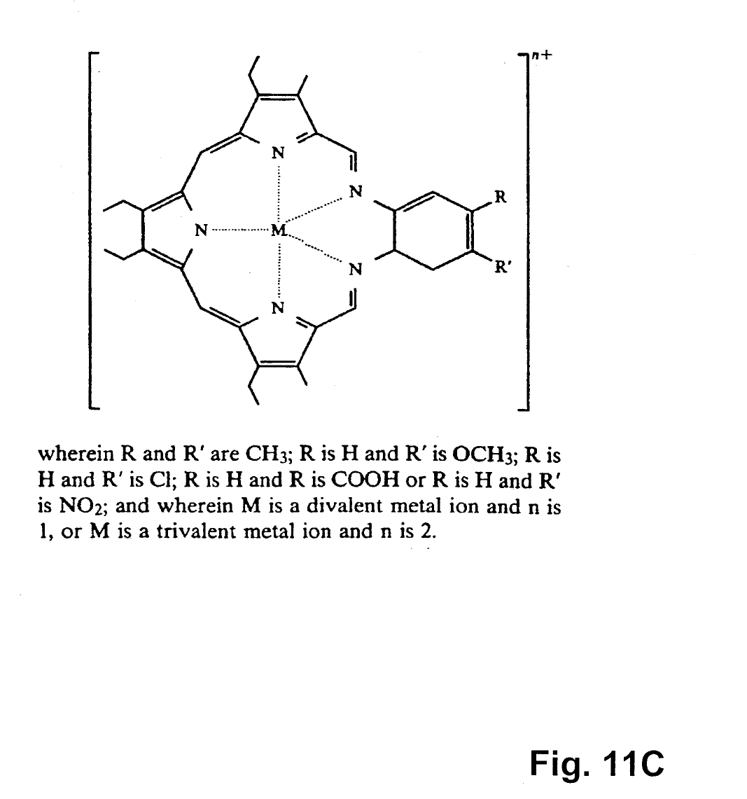

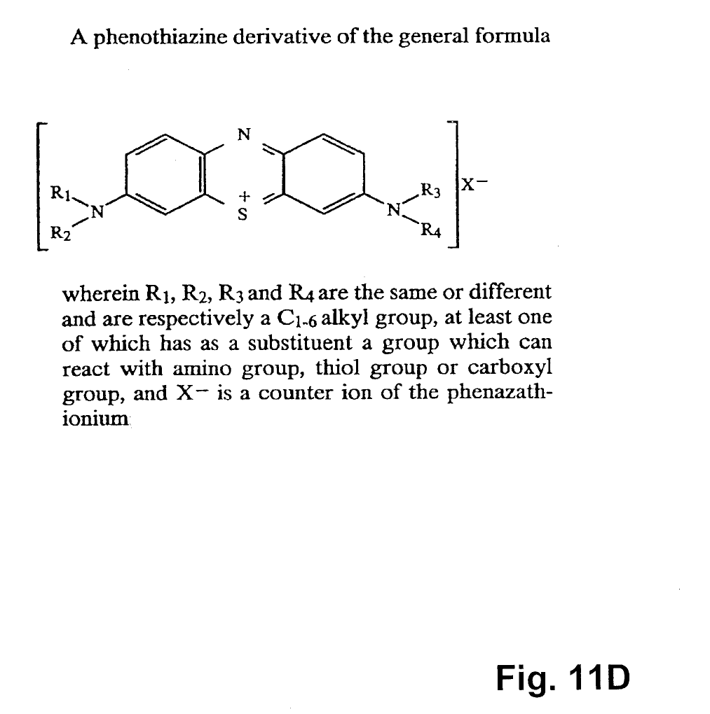

“Porphyrins” are substituted tetra-pyrrole structures in which pyrroles are coupled together with methylene bridges forming cyclic conjugated structures with chelating inner cavities. As used herein, the term porphyrin includes porphyrin derivatives, e.g. phthalocyanines and texaphyrins, that are useful in generating singlet oxygen. Representitive porphryins for use in compounds of the invention are illustrated in FIGS. 11A-C and are made as taught by Roelant, U.S. Pat. No. 6,001,573; Sagner et al, U.S. Pat. No. 6,004,530; Sessler et al, U.S. Pat. No. 5,292,414; Levy et al, U.S. Pat. No. 4,883,790; and like references.

The term “sample” in the present specification and claims is used in a broad sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

“Separation profile” in reference to the separation of molecular tags means a chart, graph, curve, bar graph, or other representation of signal intensity data versus time, or other variable related to time, that provides a readout, or measure, of the number of molecular tags of each type produced in an assay. A separation profile may be an electropherogram, a chromatogram, an electrochromatogram, or like graphical representations of data depending on the separation technique employed. A “peak” or a “band” or a “zone” in reference to a separation profile means a region where a separated compound is concentrated. There may be multiple separation profiles for a single assay if, for example, different molecular tags have different fluorescent labels having distinct emission spectra and data is collected and recorded at multiple wavelengths.

“Specific” or “specificity” in reference to the binding of one molecule to another molecule, such as a probe for a target polynucleotide, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, ionic and hydrophobic interactions, and the like, dominate the interaction of the molecules. As used herein, “stable complex” in reference to two or more molecules means that such molecules form noncovalently linked aggregates, e.g. by specific binding, that under assay conditions are thermodynamically more favorable than a non-aggregated state.

“Spectrally resolvable” in reference to a plurality of fluorescent labels means that the fluorescent emission bands of the labels are sufficiently distinct, i.e. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985).

As used herein, the term “Tm” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the T, value may be calculated by the equation. Tm=81.5+0.41 (% G+C), when a nucleic acid is in aqueous solution at I M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Terminator” means a nucleotide that can be incorporated into a primer by a polymerase extension reaction, wherein the nucleotide prevents subsequent incorporation of nucleotides to the primer and thereby halts polymerase-mediated extension. Typical terminators are nucleoside triphosphates that lack a 3′-hydroxyl substituent and include 2′,3′-dideoxyribose, 2′,3′-didehydroribose, and 2′,340 -dideoxy-3′-haloribose, e.g. 3′-deoxy-3′-fluoro-ribose or 2′,3′-dideoxy-3′-fluororibose nucleosides, for example. Alternatively, a ribofuranose analog can be used in terminators, such as 2′,3′-dideoxy-β-D-ribofuranosyl, β-D-arabinofuranosyl, 3′-deoxy-β-D-arabinofuranosyl, 3′-amino-2′,3′-dideoxy-β-D-ribofaranosyl, and 2,3′-dideoxy-3′-fluoro-β-D-ribofuranosyl. A variety of terminators are disclosed in the following references: Chidgeavadze et al., Nucleic Acids Res., 12: 1671-1686 (1984); Chidgeavadze et al., FEBS Lett., 183: 275-278 (1985); Izuta et al, Nucleosides & Nucleotides, 15: 683-692 (1996); and Krayevsky et al, Nucleosides & Nucleotides, 7: 613-617 (1988). Nucleotide terminators also include reversible nucleotide terminators, e.g. Metzker et al. Nucleic Acids Res., 22(20):4259 (1994).

### DETAILED DESCRIPTION OF THE INVENTION

The invention provides methods and compositions for generating a plurality of detection probes for one or more target nucleic acid sequences by template-dependent extension reactions. Detection probes are labeled with one or more unique molecular tags that are cleaved, separated and identified and/or quantified so that each separated molecular tag indicates the presence of an analyte in a sample and/or indicates the amount or concentration of an analyte in a sample. Extension reactions take place in the presence of a target sequence that serves as template. Extension reactions include enzymatically catalyzed polymerization, e.g. using a DNA or RNA polymerase, enzymatically catalyzed ligation, e.g. using a DNA ligase, and chemical ligation. In the formation of a detection probe by such an extension reaction, one or more molecular tags are attached to the same molecule as a cleavage-inducing moiety or a capture moiety. Either of the latter moieties permits the selective cleavage and release of molecular tags, thereby providing a measure of the amount of each target sequence in the reaction.

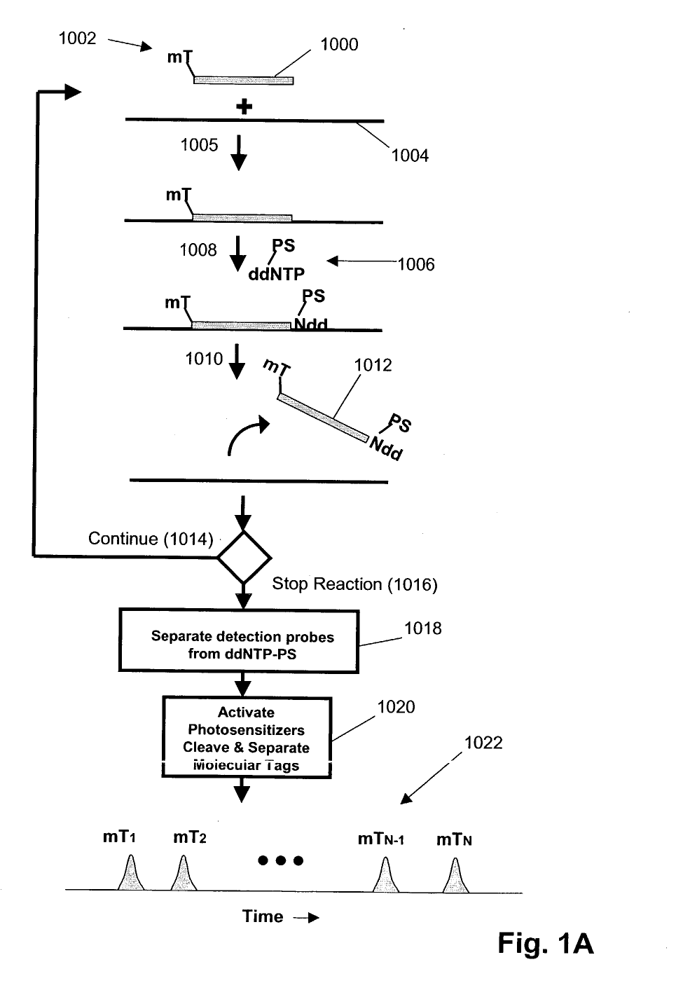

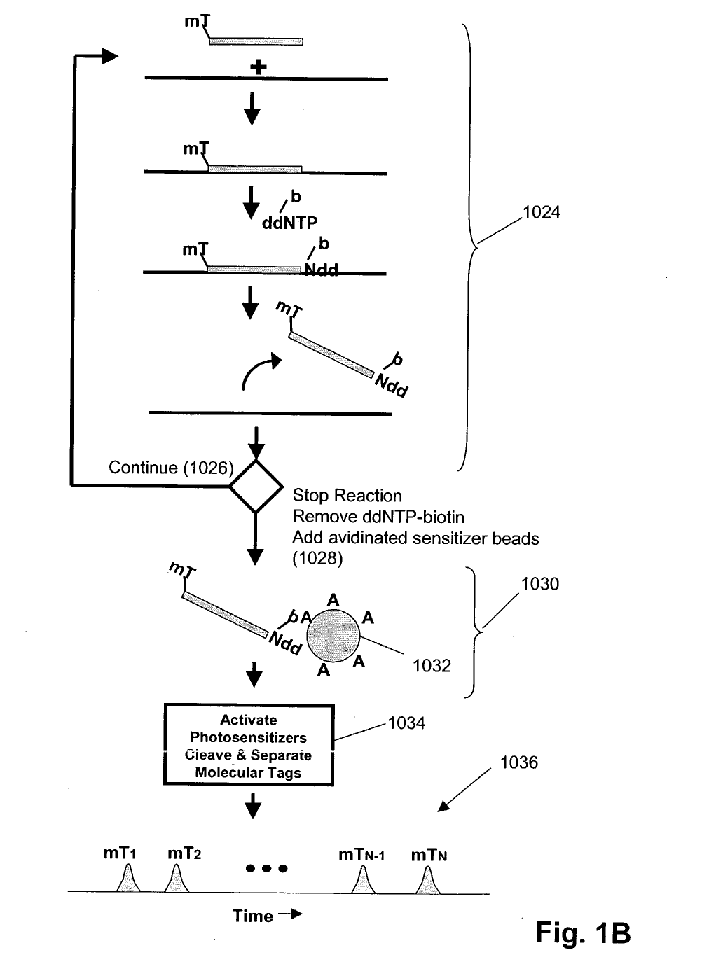

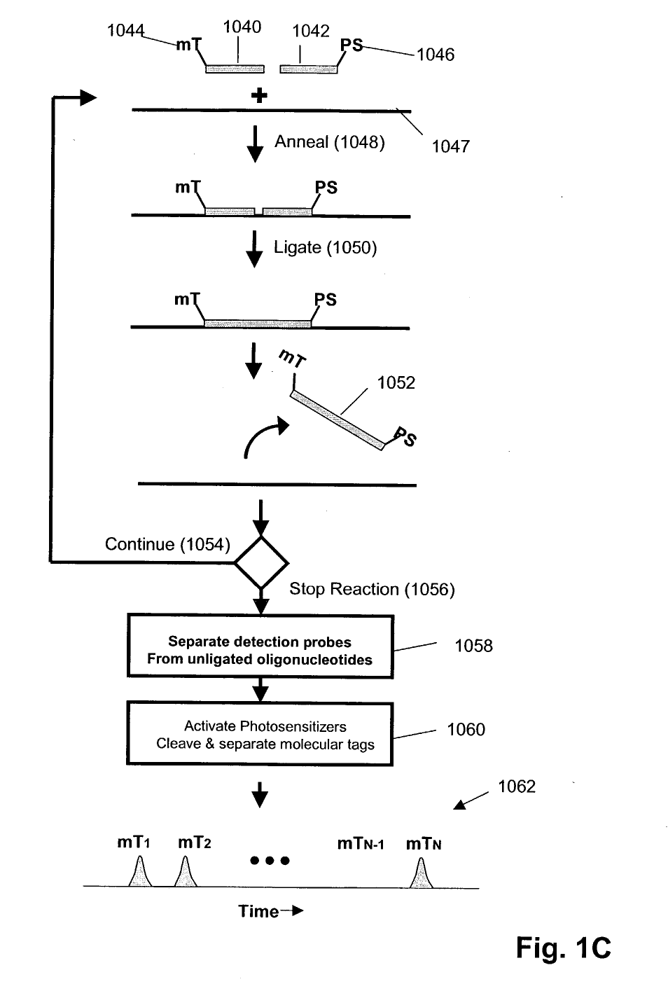

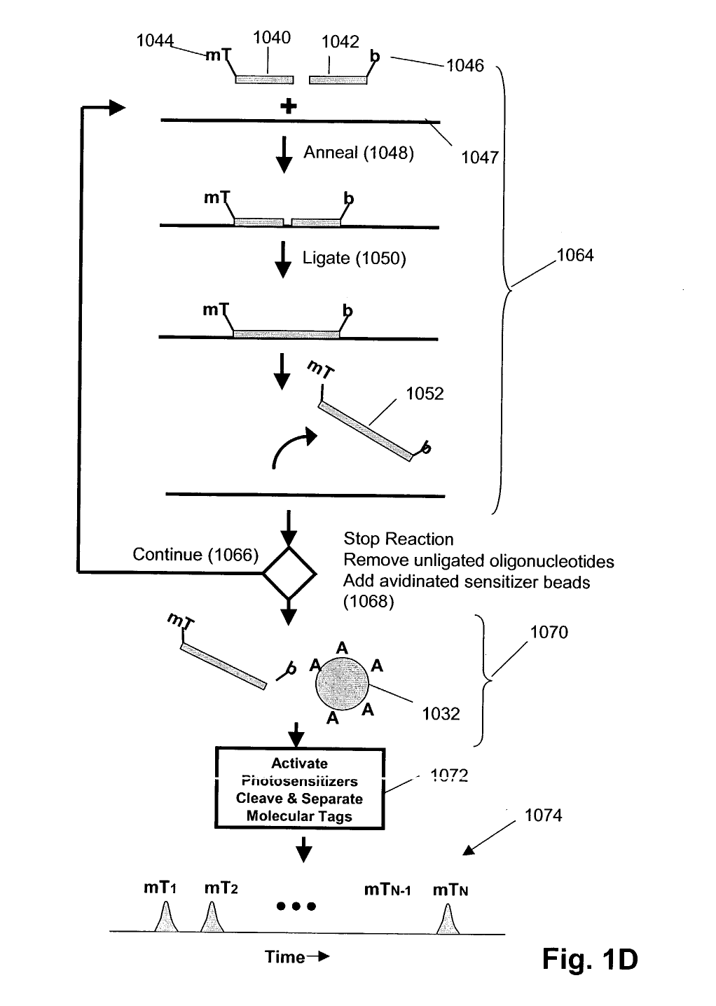

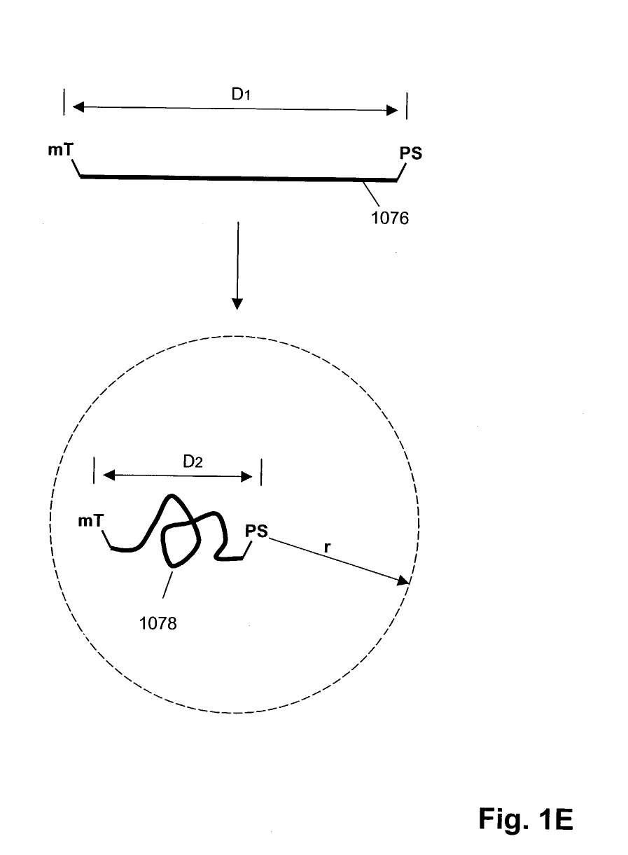

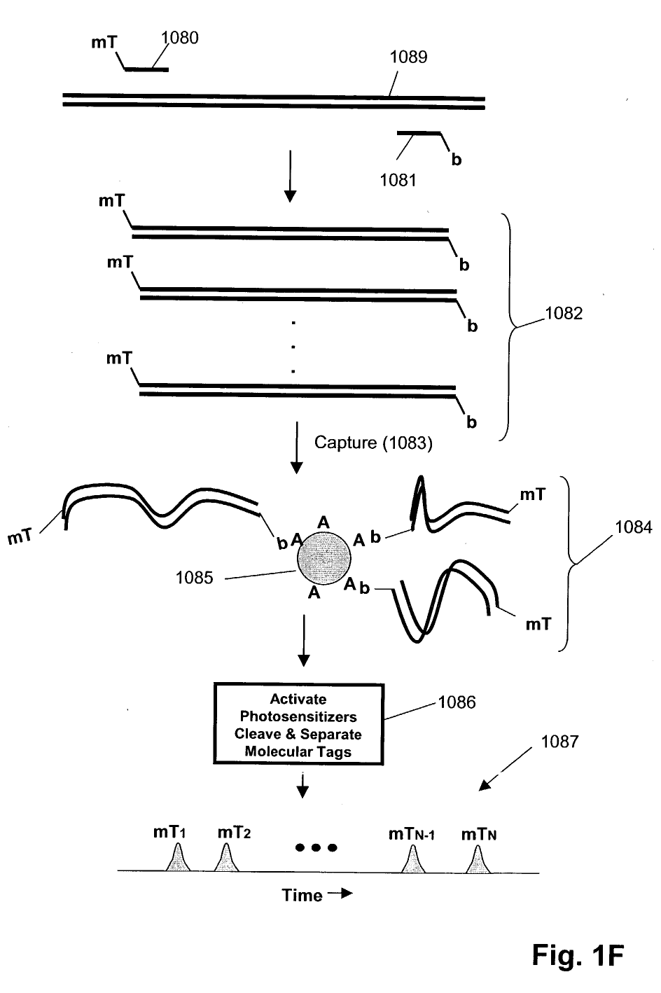

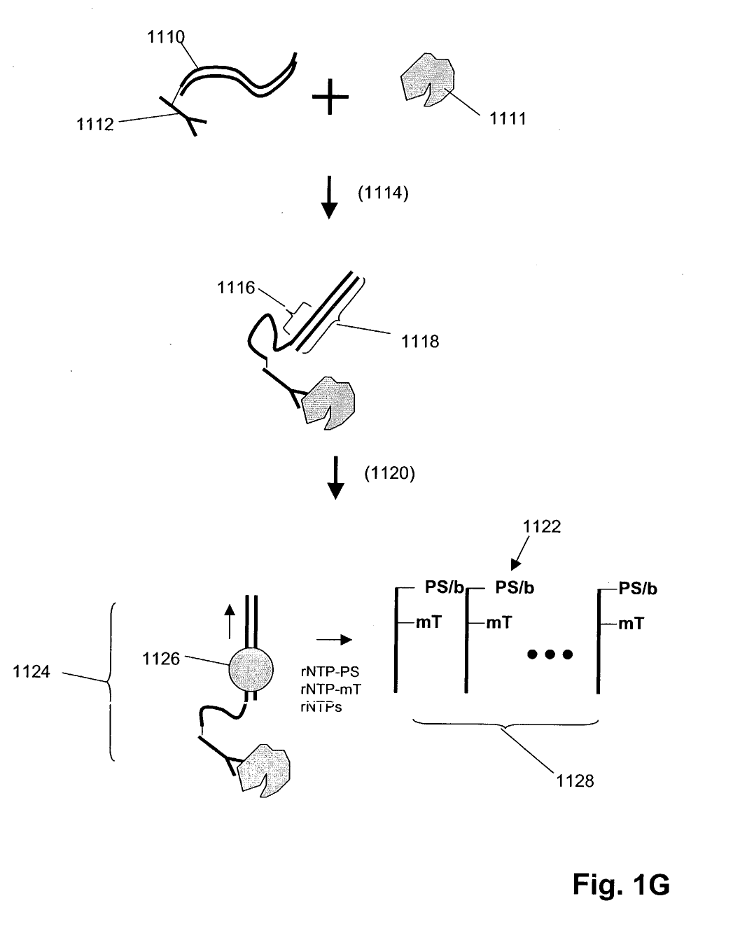

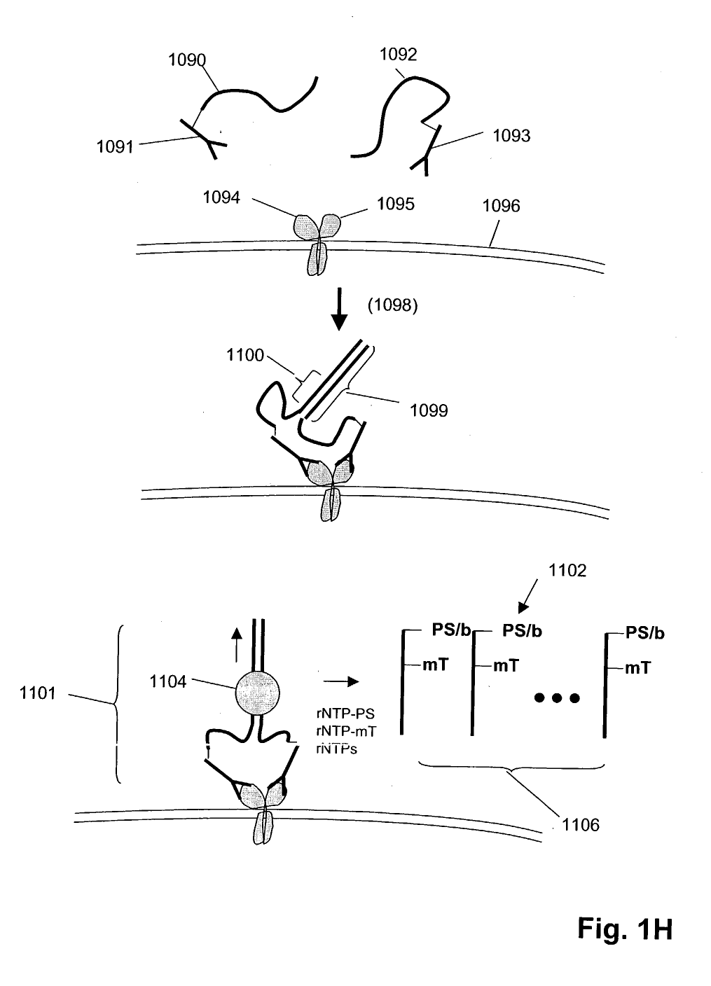

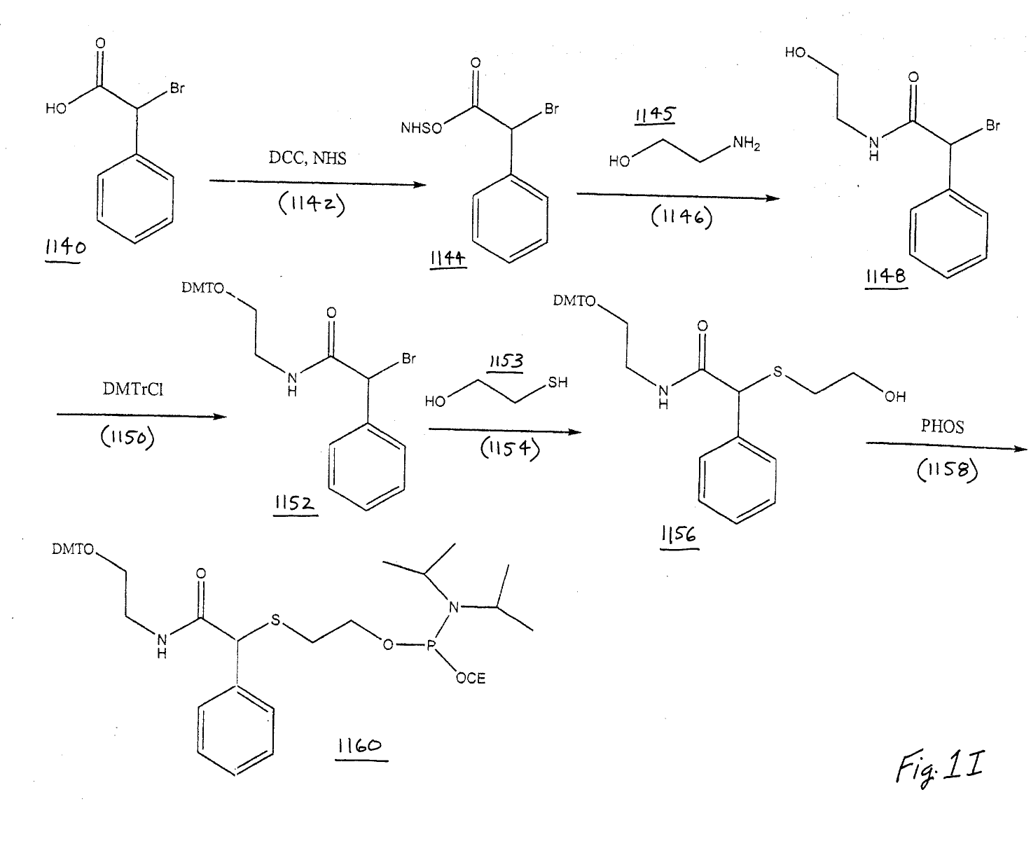

As explained more fully below, assays of the invention may be practiced in either a homogeneous format or an inhomogeneous, or nonhomogeneous, format. In the former case, a cleavage-inducing means is employed that act locally, cleaving molecular tags only within an effective proximity of each cleavage-inducing moiety. In the latter case, a separation step is included that permits a broader range of cleavage-inducing moieties to be employed. For example, the embodiments of the invention described in FIGS. 1B, 1D, 1F, 1G, and 1H are, or may be, practiced in an inhomogeneous format.

An aspect of the invention is the multiplexed detection or measurement of analytes in the same reaction. That is, in accordance with the invention, methods are provided for the simultaneous analysis of a plurality of analytes in the same reaction. The size and range of the plurality may vary from embodiment to embodiment, and conventional design trade-offs may be required for selections of particular levels of multiplexing, e.g. the sensitivity of individual analyte measurements may vary inversely with the level of multiplexing. Generally, a plurality for a given embodiment is in a range that is determined empirically using routine techniques. In one aspect, assays of the invention may detect or measure a plurality of analytes in a range of from 2 to 100, or more usually, in a range of from 2 to 50, and still more usually, in a range of from 2 to 25.

In one aspect the invention, a nucleotide conjugated to a capture moiety, a molecular tag, or a photosensitizer is added to a primer annealed to a template in a limited polymerase extension reaction. The extension reaction is limited in that the nucleotide employed is a terminator or a reduced set of nucleoside triphosphates, e.g. only dATP and dCTP, are employed so that extension stops when a template nucleotide is noncomplementary to any present in the reaction mixture. One embodiment is illustrated in FIG. 1A. Primer (1000) with molecular tag (1002) (indicated by “mT” in the figure) is annealed (1005) to template polynucleotide (1004) in the presence of a nucleic acid polymerase and one or more kinds of terminators (1006, indicated as a dideoxynucleoside terminator or “ddNTP-PS”). Each terminator is conjugated to a photosensitizer, “PS,” and usually, four terminators are employed: ddATP-PS, ddCTP-PS, ddGTP-PS, and ddTTP-PS. These compositions of the invention are described more fully below. A variety of nucleic acid polymerases may be employed depending on the nature of the template and whether RNA or DNA detection probes are desired. Nucleic acid polymerases include DNA polymerases, RNA polymerase, reverse transcriptases, and the like. As a result of the extension reaction (1008), a nucleotide is added to the 3′ end of primer (1000) to form detection probe (1012). Detection probe (1012) may be dissociated from template (1004) either by temperature cycling or by equilibrium exchange. Guidance in selecting temperatures, probe lengths, probe compositions, and like parameters, may be found in the following references which are incorporated by reference: Goelet et al, U.S. Pat. No. 6,004,744; Nikiforov et al, U.S. Pat. No. 5,679,524; Vary et al, U.S. Pat. No. 4,851,331; Hogan et al, U.S. Pat. No. 5,451,503; Western et al, U.S. Pat. No. 6,121,001; Hirschhorn et al, Proc. Natl. Acad. Sci., 97: 12164-12169 (2000); Reynaldo et al, J. Mol. Biol., 297: 511-520 (2000); Wetmur, Critical Rev. in Biochem. Mol. Biol., 26: 227-259 (1991); and the like. Usually, primers are from 12 to 25 nucleotides in length and have a Tm in the range of from 45° C. to 85° C., and more usually in the range of from 55° C. to 80° C.

Cycles of primer annealing, polymerase extension, and detection probe dissociation are continued (1014) until detectable amounts of detection probes are generated after which the reaction is stopped (1016). The reaction time required to generate detectable amounts of detection probes depends on several factors, including concentrations of reactants, whether temperature cycling or equilibrium exchange is employed, the temperatures used, the nature of the primers employed, the nature of the labels employed on the molecular tags, the separation technique employed, the sensitivity of the detection apparatus used, and the like. Selections of such parameters are routine design choices for those of ordinary skill in the art. After the reaction is stopped, detection probes are separated (1018) from the photosensitizer-labeled nucleoside triphosphates. This can be accomplished by conventional methods, e.g. QIAquick nucleotide removal kit (Qiagen, Inc., Valencia, Calif.), or like product. After the detection probes are separated from the unincorporated nucleotides, they are illuminated to activate the photosensitizers to generate singlet oxygen to release the molecular tags. The released molecular tags may be separated and identified by a variety of techniques, including electrophoresis, chromatography, mass spectroscopy, or the like. Usually, the molecular tags are separated by a liquid phase separation technique, such as chromatography or electrophoresis. Preferably, the molecular tags are separated by capillary electrophoresis, e.g. as described by Singh et al, International patent publication WO 01/83502; Singh et al, International patent publication WO 02/95356; U.S. patent publication 2002/146726; or the like.

FIG. 1B illustrates an embodiment of the invention similar to that of FIG. 1A, except that instead of incorporating a photosensitizer-labeled terminator into the primers, terminators conjugated to capture moieties, such as biotin, catechol, digoxigenin, or the like, are incorporated. Thus, cycles of annealing, polymerase extension, and dissociation (1024) are carried out in the same manner as the embodiment of FIG. 1A. Terminators conjugated to capture moieties, such as biotin, are disclosed in Ju, U.S. Pat. No. 5,876,936, which is incorporated herein by reference. As above, the reaction is continued (1026) until detectable amounts of detection probes are obtained, after which the reaction is stopped (1028), the unincorporated terminators are separated from the detection probes using conventional techniques, e.g. QIAquick Nucleotide Removal kit (Qiagen, Valencia, Calif.), or like product, and a solid phase with a capture agent attached is added. In this embodiment, a variety of cleavable linkages and cleavage moieties may be used in addition to photosensitizers, as disclosed in Singh et al, International patent publication, WO 02/95356. Exemplary reaction conditions are as follows: To a sample, a 10 μL reaction mixture is formed consisting of 80 mM Tris-HCl (pH 9.0), 2 mM MgCl₂, 100 mM primers, 3 units of AmpliTaq FS (Applied Biosystems, Foster City, Calif.), 10 μM of biotin-labeled ddNTPs. The reaction is incubated at 96° C. for 2 min followed by 30 cycles of 94° C. for 30 sec, 55° C. for 30 sec, and 72° C. for 30 sec, after which the reaction is held at 4° C. until the resulting detection probes are separated from unincorporated biotin-labeled ddNTPs.

Preferably, the solid phase supports used to capture the detection probes are streptavidinated sensitizer beads (1032), as described below and commercially available from Packard BioScience, Inc. (Meriden, Conn.), and the preferred capture moiety is biotin. This system (1030) allows convenient isolation of detection probes and generation of singlet oxygen for releasing molecular tags. After capture by sensitizer beads, the photosensitizers in the beads or attached to the beads, depending on the type of bead used, are illuminated to generate singlet oxygen that results in release of the molecular tags (1034). As above, the released molecular tags are preferably separated using conventional liquid phase separation techniques, such as capillary electrophoresis, and are identified in a separation profile (1036), usually generated based on fluorescence detection.

Besides extension with a nucleic acid polymerase, a primer of the invention may also be extended by enzymatic or chemical ligation, as illustrated by the embodiment of FIG. 1C. In this embodiment, primer (1040) having molecular tag (1044) attached is combined with oligonucleotide (1042) having photosensitizer (1046) attached and a sample containing template sequence (1047). Primer (1040) and oligonucleotide (1042) are designed to form perfectly matched duplexes with template sequence (1047) under assay conditions whenever template sequence (1047) is present. In embodiments wherein enzymatic ligation is used, oligonucleotide (1042) has a 5′ phosphate, and usually, a 3′ blocking group, e.g. a phosphate, dideoxynucleotide, or the like, to prevent spurious ligations. This embodiment may be employed to monitor gene expression or to detect the presence of sequence polymorphisms, such as single nucleotide polymorphisms, e.g. Landegren and Hood, U.S. Pat. No. 4,988,617; Whitely et al, U.S. Pat. No. 5,521,065; Eggerding, U.S. Pat. No. 6,130,073; Schouten et al, Nucleic Acids Research, 30: e57 (2002); and Taylor et al, Biotechniques, 30:661-669 (2001), which references are incorporated by reference. As exemplified in the foregoing references, assays for detection and monitoring using ligation extension may have various forms including, but not limited to (i) ligation-based amplification of a template sequence prior to detection, (ii) amplification by polymerase chain reaction (PCR) prior to detection by a ligation reaction, (iii) assay specificity may be controlled by the thermodynamics of primer and oligonucleotide annealing and/or by the ability of a ligase to distinguish adjacent oligonucleotide and primer that are perfectly complementary with a template from those containing mismatches at or near the junction between the oligonucleotide and primer, (iv) detection probe accumulation by equilibrium exchange, and the like. Selection of a particular format involves conventional design choices within the skill of ordinary practitioners of the art. Ligation can be accomplished either enzymatically or chemically. Chemical ligation methods are well known in the art, e.g. Ferris et al, Nucleosides & Nucleotides, 8: 407-414 (1989); Shabarova et al, Nucleic Acids Research, 19: 4247-4251 (1991); and the like. Preferably, enzymatic ligation is carried out using a ligase in a standard protocol. Many ligases are known and are suitable for use in the invention, e.g. Lehman, Science, 186: 790-797 (1974); Engler et al, DNA Ligases, pages 3-30 in Boyer, editor, The Enzymes, Vol. 15B (Academic Press, New York, 1982); and the like. Preferred ligases include T4 DNA ligase, T7 DNA ligase, E. coli DNA ligase, Taq ligase, Pfu ligase, and Tth ligase. Protocols for their use are well known, e.g. Sambrook et al (cited above); Barany, PCR Methods and Applications, 1: 5-16 (1991); Marsh et al, Strategies, 5: 73-76 (1992); and the like. Generally, ligases require that a 5′ phosphate group be present for ligation to the 3′ hydroxyl of an abutting strand.

Returning to FIG. 1C, when primer (1040) and oligonucletide (1042) anneal and form perfectly matched duplexes with template (1047), they are covalently joined or ligated (1050) in a reaction that usually creates a phosphodiester bond between them. In embodiments that employ chemical ligation, other types of linkages or bonds may be formed, e.g. Letsinger and Gryaznov, U.S. Pat. No. 5,476,930. As above, depending on the assay format selected, the reaction may be subjected to temperature cycling or simply equilibrium exchange (1054) in order to accumulate a detectable amount of detection probe (1052). After a detectable amount of detection probe (1052) has been accumulated, the reaction is stopped (1056) and detection probes (1052) are separated (1058) from unligated primers (1040) and oligonucleotides (1042). Such separation may be carried out by any convenient method, such as electrophoresis, chromatography, or the like, after which the isolated detection probes are treated with a cleavage agent to cleave and release the molecular tags, e.g. with illumination if the cleavage agent is a photosensitizer that generates singlet oxygen (1060). After such cleavage, the released molecular tags are separated and identified (1062).

In regard to the embodiments of FIGS. 1A and 1C, the lengths of the detection probes may vary widely. One design constraint is that the cleavable linkage of the molecular tag be within the effective proximity of the photosensitizer. As shown in FIG. 1E, the distance D1 between a photosensitizer and a cleavable linkage may greatly exceed the effective proximity of the photosensitizer when the detection probe (1076) is fully hybridized to a template. After dissociation, the detection probe forms a random coil (1078) and the average distance between the cleavable linkage and the photosensitizer is reduced, preferably so that the average distance, D2 is within the effective proximity r (1077) of the photosensitizer.

As with the embodiment of FIG. 1B, the photosensitizer (1046) of oligonucleotide (1042) may be replaced with a capture moiety, such as biotin (1043), as shown in FIG. 1D. The ligation reaction is carried out (1064) as described for the embodiment of FIG. 1C. After continuing (1066) the reaction until a detectable amount of detection probe (1052) is accumulated, the reaction is stopped (1068), unligated primer and biotinylated oligonucleotide are removed, or separated, from detection probe, and the detection probe is captured (1070) on avidinated beads, or like solid phase support. As explained above, a variety of cleavable linkages and cleavage methods may be used in this embodiment, which is in a so-called inhomogeneous format. Preferably, the cleavable linkage between the molecular tag and the detection probe may be cleaved by oxidation and the cleavage-inducing moiety is a sensitizer that generates singlet oxygen. More preferably, the cleavage inducing moiety is a photosensitizer, and still more preferably, the photosensitizer is carried on or in a photosensitizer bead, e.g. as disclosed below. In such an embodiment, the photosensitizers in the photosensitizer beads are activated (1072) by illumination so that the molecular tags are released, after which they are separated (1074) so that distinguishable peaks or bands are formed in a separation profile, such as an electropherogram or chromatogram.

The invention may also be practiced with PCR, as exemplified in the embodiment illustrated in FIG. 1F. A segment of target polynucleotide (1089) is amplified using primer (1080) having a molecular tag attached and primer (1081) having a capture moiety attached, such as biotin, to form amplicon (1082). After separation or removal of the unincorporated primers, amplicon (1082) is captured (1083) with a solid phase, such as beads, derivatized with a capture agent that binds the capture moiety. As discussed above, many different cleavable linkages and cleavage-inducing moieties may be employed. Usually, cleavable linkages are oxidation labile and cleavage-inducing moieties generate singlet oxygen. Preferably, cleavage-inducing moieties comprise photosensitizer beads. After capture, the photosensitizers are activated so that singlet oxygen is produced and the molecular tags are released and separated (1086) so that distinct peaks are formed in a separation profile (1087).

Another embodiment of the invention is illustrated in FIG. 1G wherein the generation of detection probes indicates or signals the occurrence of a binding event. Double stranded polynucleotide (1110) is covalently attached to antibody (1112) using conventional techniques, e.g. Hermanson, Bioconjugate Techniques (Academic Press, New York, 1996), or the like. Double stranded region (1118) of polynucleotide (1110) contains RNA polymerase recognition site (1116) and a sequence that serves as a label for the antibody to which it is attached. After antibody (1112) specifically binds to analyte (1111) and unbound antibody is removed, e.g. by washing, an RNA polymerase (1126) is added (1114) in the presence of ribonucleoside triphosphates under reaction conditions (1120) that permit it to bind to recognition site (1116) and generate detection probes (1128). Detection probes (1128) are formed by the incorporation of a molecular tag-labeled ribonucleoside triphosphate (shown as “rNTP-mT”) and the incorporation of a capture moiety-labeled or photosensitizer-labeled ribonucleoside triphosphate (“rNTP-PS”) (1122) during synthesis by RNA polymerase (1126). Exemplary RNA polymerases include, but are not limited to, T7 RNA polymerase and T3 RNA polymerase. The length of polynucleotide (1110) may vary widely. In one aspect, it is in the range of from about 20 nucleotide pairs to about 100 nucleotide pairs, and it is attached to antibody (1112) in an orientation (1124) such that RNA polymerase (1126) binds to a recognition site proximal to antibody (1112) and progresses in a distal direction from antibody (1112) as it incorporates ribonucleoside triphosphates. The location of incorporation of molecular tags, capture moieties, and photosensitizers may be controlled by selection of the sequence of polynucleotide (1110). For example, the following polynucleotide (SEQ ID NO: 1) contains in series spacer nucleotides (a's and c's), T7 recognition site, four spacer nucleotides (a's and c's), a single “t” for incorporation of a molecular tag, eight spacer nucleotides (a's and c's), and a single “g” for incorporation of a capture moiety, such as biotin (“b”) or a photosensitizer.

```
5′-accaccaccctaatacgactcactatagggaccataccaaccag
      tggtggtgggattatgctgagtgatatccctggtatggttggtc
                                          ↑ ↑ ↑
                      T7 RecognitionSite mT capture moiety
                                                              incorporation incorporation
```

Up to four-fold multiplexing is possible in this embodiment as each different molecular tag is attached to a different one of riboadenosine triphosphate, riboguanosine triphosphate, ribocytidine triphosphate, or ribothymidine triphosphate. In the presence of an appropriate RNA polymerase (1126), complex (1124) generates detection probes (1128) that are each labeled with a molecular tag (“mT”) and either a photosensitizer or capture moiety, such as biotin (1122). Molecular tags, photosensitizers, or capture moieties are incorporated into a detection probe as labels on ribonucleoside triphosphates. Such labeled ribonucleoside triphosphates are described more fully below, and Sasaki et al, Proc. Natl. Acad. Sci., 95: 3455-3460 (1998), provides guidance for selecting reaction conditions, RNA polymerase, and the like, for such extension reactions. After detection probes (1128) are generated, they are separated from unincorporated ribonucleoside triphophates, molecular tags are released as described above, and the released molecular tags are separated, e.g. by electrophoresis, and identified.

Another embodiment of the invention employing antibody binding compositions is illustrated in FIG. 1H. In this embodiment, two antibody binding compositions provide greater sensitivity and permit the measurement of multi-component analytes, such as receptor homodimers or heterodimers. First polynucleotide (1090) is covalently attached to first antibody binding composition, or first antibody (1091), which is specific for a first analyte, such as receptor component (1094) in surface membrane (1096). Likewise, second polynucleotide (1092) is covalently attached to second antibody binding composition, or second antibody (1093), specific for a second analyte. For example, as illustrated in FIG. 1H, the first and second analytes may be components of a receptor heterodimer, separate epitopes on the same protein, or the like. Polynucleotides (1090) and (1092) may be attached to their respective antibodies at either a 5′ end or a 3′ end; however, usually, one is attached at a 5′ end and the other is attached at a 3′ end so a duplex (1099) with a free end is formed. When antibodies (1094) and (1095) bind to their respective antigens and the antigens are sufficiently close to one another, such as receptor components forming a heterodimer, portions of polynucleotides (1090) and (1092) are able to form a perfectly matched duplex with one another (1098). The sequence of the complementary region (1099) is designed to include a recognition site (1100) for an RNA polymerase. In the presence of an appropriate RNA polymerase (1104), complex (1101) generates detection probes (1106) that are labeled with molecular tag (“mT”) and either a photosensitizer or capture moiety, such as biotin (1102). After such generation, the detection probes are processed as described above to produce a separation profile from which an assay readout is obtained.

### Assay Compositions

A. Primers with Molecular Tags or Photosensitizers Attached.

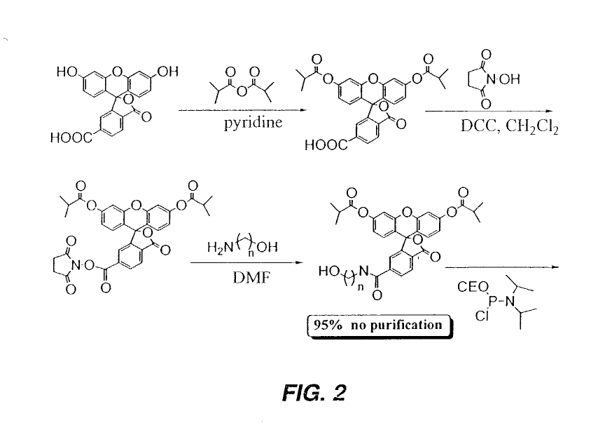

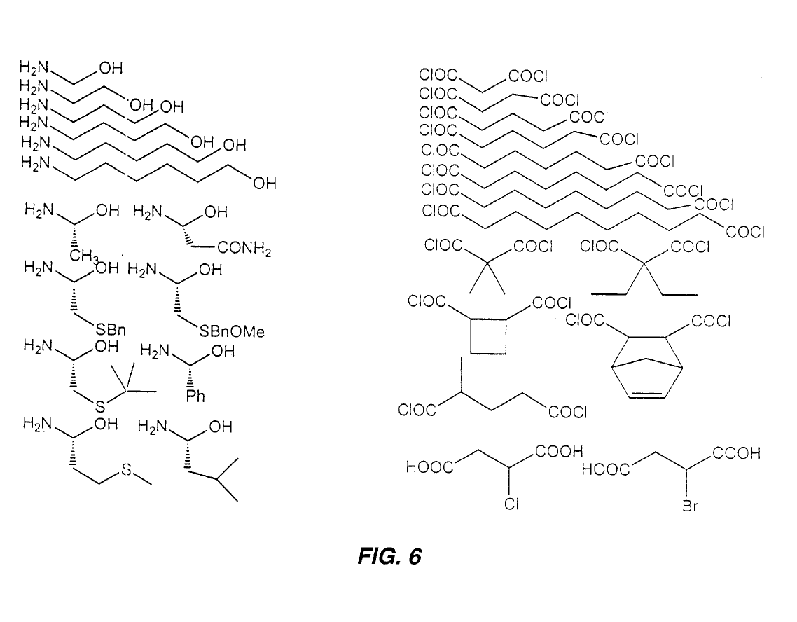

Molecular tags or photosensitizer molecules may be attached to a variety of locations on a primer, including bases, sugars, or phosphate groups using known chemistries, e.g. Hermanson (cited above). Such labels are conveniently attached to the 5′ end of an oligonucleotide to form a primer of the invention. The attachment may be carried out as the final coupling steps in the synthesis of a primer on a conventional solid phase DNA synthesizer, or the attachment may be carried out after solid phase synthesis of an oligonucleotide that includes the coupling of a reactive functionality, such as a free amine, as the final coupling step. In the former case, the molecular tag or photosensitizer may be assembled using phosphoramidite derivatives of the photosensitizer or molecular tag, or or components thereof. For example, a phosphoramidite reagent for assembling a molecular tag on a primer is illustrated in FIG. 1I. This reagent introduces a thioether linkage between the oligonucleotide of the primer and a molecular tag, and may be cleaved by oxidation to release a molecular tag. α-bromophenylacetic acid (1140) is reacted (1142) with N-hydroxysuccinimide (NHS) and dicyclohexylcarbodiimide (DCC) to give NHS ester product (1144), which is then reacted (1146) with hydroxylamine (1145) to give compound (1148). The free hydroxyl of compound (1148) is protected (1150) by reacting with dimethyltrityl chloride to give compound (1152), which is then reacted (1154) with hydroxythiol (1153) to give compound (1156). The free hydroxyl of compound (1156) is then phosphitylated (1158) to give DMT-protected phosphoramidite (1160). This reagent may be used with the fluorescein phosphoramidites disclosed in FIGS. 2-6 to complete the synthesis of primers having cleavable molecular tags.

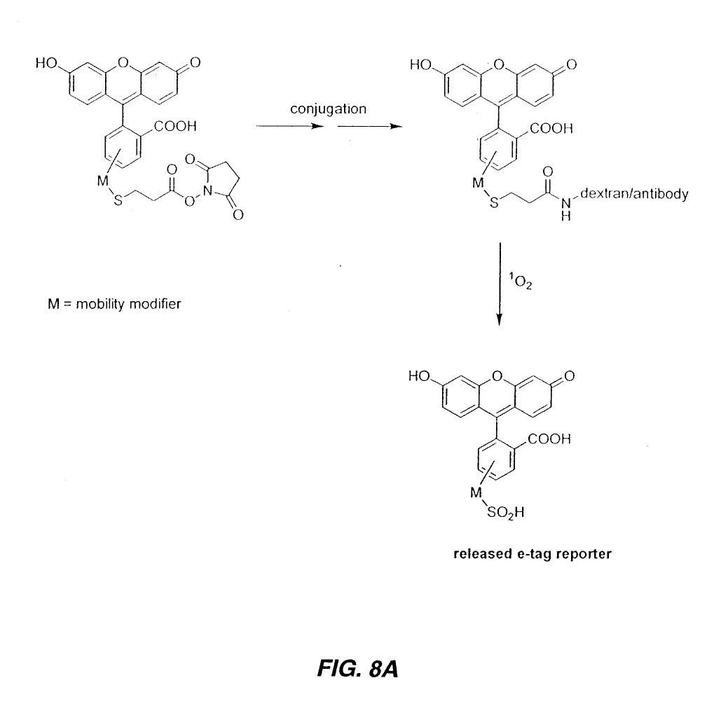

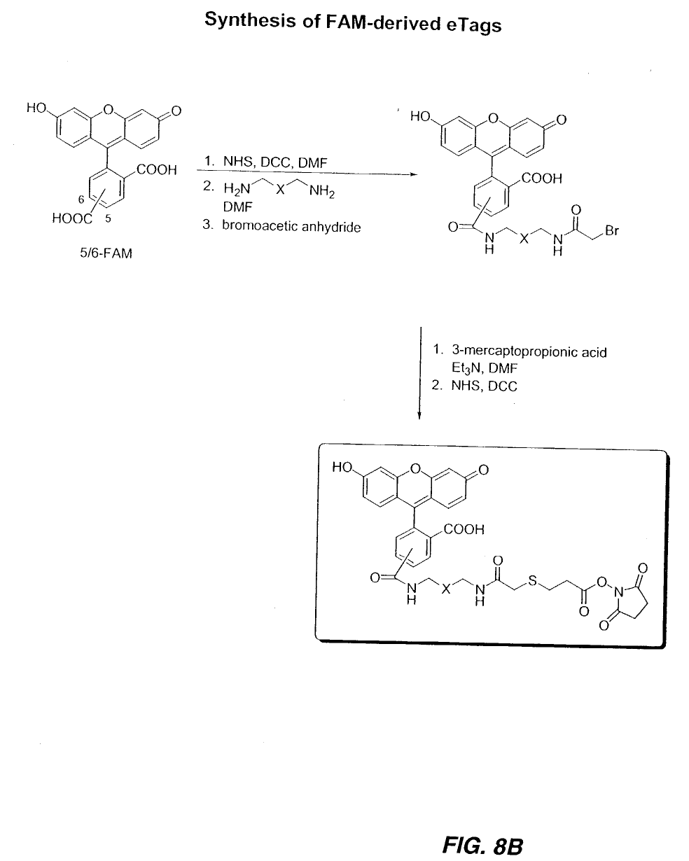

Alternatively, primer having either molecular tags or photosensitizers may be synthesized by first making an oligonucleotide having a 5′ functionality, such as a free amine. A free amine is conveniently added as a final step in solid phase synthesis by using a reagent such as AminoLink™ (Applied Biosystems, Foster City, Calif.), disclosed in Fung et al, U.S. Pat. No. 4,757,141. NHS esters of molecular tags or photosensitizers are then conveniently coupled to the free amine of the oligonucleotide using conventional reaction conditions to form a primer of the invention. NHS esters of molecular tags are disclosed below and in FIGS. 8A-B, 9A-I, and 10A-I. NHS esters of a wide range of porphyrin photosensitizers are disclosed in Roelant, U.S. Pat. No. 6,001,573; Sagner et al, U.S. Pat. No. 6,004,530; Motsenbocker, U.S. Pat. No. 5,532,171; and Masuya et al, U.S. Pat. No. 5,344,928, which patents are incorporated by reference. Magda et al, U.S. Pat. No. 5,565,552 which is incorporated by reference, discloses the direct attachment of porphyrin photosensitizers to oligonucleotides during solid phase synthesis using porphyrin phosphoramidite intermediates.

B. Nucleoside Triphosphates with Photosensitizers or Capture Moieties Attached.

Compositions of the invention include nucleoside triphosphates derivatized with a photosensitizer for enzymatic incorporation into detection probes by a nucleic acid polymerase. In one aspect, such compounds of the invention are defined by the following formula:

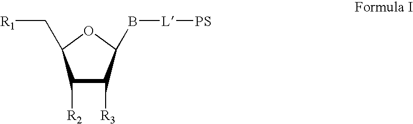

wherein B is a nucleobase, L′ is a linker, PS is a photosensitizer, R₁ is —OH, or mono-, di-, or triphosphate, or an analog thereof, such as phosphorothioate, phosphoramidate, or the like; R₂ is —OH or a group that prevents further extension of a primer, such as H, F, Cl, NH₂, N₃, or OR′ where R′ is C1-C6 alkyl; R₃ is —OH, H, F, Cl, NH₂, N₃, or OR′ where R′ is C1-C6 alkyl. In one aspect, R₁ is triphosphate, and R₂ and R₃ are each H. In another aspect, R₁ is triphosphate, and R₂ is —OH and R₃ is H. In another aspect, R₁ is triphosphate, and R₂ is H and R₃ is —OH. Exemplary nucleobases include adenine, 7-deazaadenine, 7-deaza-8-azaadenine, cytosine, guanine, 7-deazaguanine, 7-deaza-8-azaguanine, thymine, uracil, and inosine. Nucleobase B is attached to the C1 carbon of the sugar moiety as with natural nucleosides. In one aspect, PS is a porphryin, phthalocyanine, or a thiazine dye, such as disclosed by Roelant, U.S. Pat. No. 6,001,573; Sagner et al, U.S. Pat. No. 6,004,530; Sessler et al, U.S. Pat. No. 5,292,414; Levy et al, U.S. Pat. No. 4,883,790; Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Motsenbocker, U.S. Pat. No. 5,532,171; and Masuya et al, U.S. Pat. No. 5,344,928, which patents are incorporated by reference. Exemplary photosensitizers for use in the invention are illustrated in FIGS. 11A-D Generally, PS is coupled via linker, L′, to B by way of conventional attachment sites, e.g. 4-position of cytosine, 6-position of adenosine, 5-position of pyrimidines, 8-position of purines, and 7-position of 7-deazapurines. Linker, L′, may have a wide variety of forms. In one aspect, the terminal moiety of L′ nearest B is an acetylene moiety (—C≡C—) or a propargyl moiety (—C≡CCH₂—), since such linkage moieties tend to be particularly compatible with a variety of polymerases using in primer extension. Other non-acetylenic-based linkers are also contemplated. Exemplary linkers are disclosed in Hobbs et al, U.S. Pat. Nos. 5,151,507; and 5,047,519; Kahn et al, U.S. Pat. Nos. 5,821,356; 5,770,716; 5,948,648; 6,096,875; Benson et al, U.S. Pat. Nos. 5,936,087; and 6,008,379; Lee et al, U.S. Pat. Nos. 6,080,852; and 6,080,852; which patents are incorporated by reference. In one aspect, L′ is “—C≡C—W₁—NH—,” wherein W₁ is a substituted or unsubstituted diradical moiety of from 1 to about 30 atoms. W₁ can be straight-chained alkylene, C1-C20, optionally containing within the chain double bonds, triple bonds, aryl groups or heteroatoms, such as N, O, or S. Exemplary linkers include the following diradical moieties:

—C≡CCH₂—NH—

—C≡CCH₂—OCH₂CH₂NH—

—C≡CCH₂—OCH₂CH₂OCH₂CH₂NH—

—C≡CCH₂—NHC(O)(CH₂)₅NH—

—C≡CC(O)NH(CH₂)₅NH—

—C≡C-(p-C₆H₄)—OCH₂CH₂NH—

—C≡C-(p-C₆H₄)-(p-C₆H₄)—C≡C—

—C≡CCH₂—(O—CH₂—CH₂)ₙ—NH—, where n=1, 2, or 3.

Preferably, nucleotides derivatized with such linkers with free amines are reacted with NHS esters of a porphryin to form a compound of Formula I.

C. Nucleoside Triphosphates with Molecular Tags Attached.

Compositions of the invention include nucleoside triphosphates derivatized with a molecular tag for enzymatic incorporation into detection probes by a nucleic acid polymerase. In one aspect, such compounds of the invention are defined by the following formula:

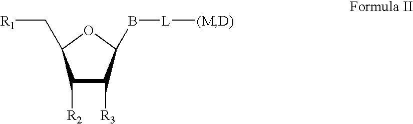

wherein B is a nucleobase, L is a cleavable linkage, —(M,D) is a molecular tag where M is a mobility modifier and D is a detectable moiety described more fully below, R₁ is —OH, or mono-, di-, or triphosphate, or an analog thereof, such as phosphorothioate, phosphoramidate, or the like; R₂ is —OH or a group that prevents further extension of a primer, such as H, F, Cl, NH₂, N₃, or OR′ where R′ is C1-C6 alkyl; R₃ is —OH, H, F, Cl, NH₂, N₃, or OR′ where R′ is C1-C6 alkyl. In one aspect, R₁ is triphosphate, and R₂ and R₃ are each H. In another aspect, R₁ is triphosphate, and R₂ is —OH and R₃ is H. In another aspect, R₁ is triphosphate, and R₂ is H and R₃ is —OH. Exemplary nucleobases include adenine, 7-deazaadenine, 7-deaza-8-azaadenine, cytosine, guanine, 7-deazaguanine, 7-deaza-8-azaguanine, thymine, uracil, and inosine. Nucleobase B is attached to the C1 carbon of the sugar moiety as with natural nucleosides. Generally, a molecular tag is coupled via a cleavable linkage to B by way of conventional attachment sites, e.g. 4-position of cytosine, 6-position of adenosine, 5-position of pyrimidines, 8-position of purines, and 7-position of 7-deazapurines. Cleavable linkage, L, may have a wide variety of forms. In one aspect, cleavable linkage, L, is formed from linker L′ (described above). As above, preferably, the terminal moiety of L′ nearest B is an acetylene moiety (—C≡C—) or a propargyl moiety (—C≡CCH₂—). Other non-acetylenic-based linkers are also contemplated. Exemplary linkers are disclosed in Hobbs et al, U.S. Pat. Nos. 5,151,507; and 5,047,519; Kahn et al, U.S. Pat. Nos. 5,821,356; 5,770,716; 5,948,648; 6,096,875; Benson et al, U.S. Pat. Nos. 5,936,087; and 6,008,379; Lee et al, U.S. Pat. Nos. 6,080,852; and 6,080,852; which patents are incorporated by reference. Exemplary linkers from which a cleavable linkage, L, is formed include the same diradical moieties listed above. Preferably, the free amines of nucleotides derivatized with such linkers are reacted with NHS esters of a molecular tag (described below) to form a compound of Formula II.

D. Polynucleotides Attached to Antibody Binding Compositions.

Polynucleotides are attached to antibody binding compositions by either their 5′ ends or 3′ ends using conventional chemistries disclosed in the following references which are incorporated by reference: Fung et al (cited above), Hermanson (cited above), Mullah et al, U.S. Pat. No. 5,736,626; Nelson, U.S. Pat. No. 5,401,837; Sano et al, U.S. Pat. No. 5,665,539; Dattagupta et al, U.S. Pat. No. 4,748,111; Nilsen, U.S. Pat. No. 6,117,631; Martinelli et al, U.S. Pat. No. 6,083,689; and the like.

### Molecular Tags

In one embodiment, molecular tags are cleaved from a detection probe by reaction of a cleavable linkage with an active species, such as singlet oxygen, generated by a cleavage-inducing moiety, e.g. Singh et al, International patent publication WO 01/83502. A cleavable linkage can be virtually any chemical linking group that may be cleaved under conditions that do not degrade the structure or affect detection characteristics of the released molecular tag. Whenever compositions of the invention are used in a homogeneous assay format, the cleavable linkage holding a molecular tag to a detection probe is cleaved by a cleavage agent that acts over a short distance so that only cleavable linkages in its immediate proximity are cleaved. Typically, such an agent must be activated by making a physical or chemical change to the reaction mixture so that the agent produces an short lived active species that diffuses to a cleavable linkage to effect cleavage.

In a non-homogeneous, or heterogeneous format, detection probes are separated from primers or unincorporated nucleoside triphosphates. Thus, a wider selection of cleavable linkages and cleavage agents are available for use with the invention. Cleavable linkages may not only include linkages that are labile to reaction with a locally acting reactive species, such as singlet oxygen, but also include linkages that are labile to agents that operate throughout a reaction mixture, such as a base cleaving all base-labile linkages, general illumination by light of an appropriate wavelength cleaving all photocleavable linkages, and so on. Additional linkages cleavable by agents that act generally throughout a reaction mixture include linkages cleavable by reduction, linkages cleaved by oxidation, acid-labile linkages, peptide linkages cleavable by specific proteases, and the like. References describing many such linkages include Greene and Wuts, Protective Groups in Organic Synthesis, Second Edition (John Wiley & Sons, New York, 1991); Hermanson, Bioconjugate Techniques (Academic Press, New York, 1996); and Still et al, U.S. Pat. No. 5,565,324.

When L is oxidation labile, L is preferably a thioether or its selenium analog; or an olefin, which contains carbon-carbon double bonds, wherein cleavage of a double bond to an oxo group, releases the molecular tag, —(M,D). Illustrative olefins include vinyl sulfides, vinyl ethers, enamines, enamines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photo-activation in the absence or presence of a photosensitizer. Such reactions are described in the following exemplary references: Adam and Liu, J. Amer. Chem. Soc. 94, 1206-1209, 1972, Ando, et al., J.C.S. Chem. Comm. 1972, 477-8, Ando, et al., Tetrahedron 29, 1507-13, 1973, Ando, et al., J. Amer. Chem. Soc. 96, 6766-8, 1974, Ando and Migita, ibid. 97, 5028-9, 1975, Wasserman and Terao, Tetra. Lett. 21, 1735-38, 1975, Ando and Watanabe, ibid. 47, 4127-30, 1975, Zaklika, et al., Photochemistry and Photobiology 30, 35-44, 1979, and Adam, et al., Tetra. Lett. 36, 7853-4, 1995. See also, U.S. Pat. No. 5,756,726.

The formation of dioxetanes is obtained by the reaction of singlet oxygen with an activated olefin substituted with an molecular tag at one carbon atom and the binding moiety at the other carbon atom of the olefin. See, for example, U.S. Pat. No. 5,807,675. These cleavable linkages may be depicted by the following formula:

—W—(X)ₙC_{α}=C_{β}(Y)(Z)—

wherein:

W may be a bond, a heteroatom, e.g., O, S, N, P, M (intending a metal that forms a stable covalent bond), or a functionality, such as carbonyl, imino, etc., and may be bonded to X or C_{α}, at least one X will be aliphatic, aromatic, alicyclic or heterocyclic and bonded to C_{α} through a hetero atom, e.g., N, O, or S and the other X may be the same or different and may in addition be hydrogen, aliphatic, aromatic, alicyclic or heterocyclic, usually being aromatic or aromatic heterocyclic wherein one X may be taken together with Y to form a ring, usually a heterocyclic ring, with the carbon atoms to which they are attached, generally when other than hydrogen being from about 1 to 20, usually 1 to 12, more usually 1 to 8 carbon atoms and one X will have 0 to 6, usually 0 to 4 heteroatoms, while the other X will have at least one heteroatom and up to 6 heteroatoms, usually 1 to 4 heteroatoms;

Y will come within the definition of X, usually being bonded to C_{β} through a heteroatom and as indicated may be taken together with X to form a heterocyclic ring;

Z will usually be aromatic, including heterocyclic aromatic, of from about 4 to 12, usually 4 to 10 carbon atoms and 0 to 4 heteroatoms, as described above, being bonded directly to C_{β} or through a heteroatom, as described above;

n is 1 or 2, depending upon whether the molecular tag is bonded to C_{α}or X;

wherein one of Y and Z will have a functionality for binding to the binding moiety, or be bound to the binding moiety, e.g. by serving as, or including a linkage group, to a binding moiety, T.

Preferably, W, X, Y, and Z are selected so that upon cleavage molecular tag, E, is within the size limits described below.

Illustrative cleavable linkages include S(molecular tag)-3-thiolacrylic acid, N(molecular tag), N-methyl 4-amino4-butenoic acid, 3-hydroxyacrolein, N-(4-carboxyphenyl)-2-(molecular tag)-imidazole, oxazole, and thiazole.

Also of interest are N-alkyl acridinyl derivatives, substituted at the 9 position with a divalent group of the formula:

—(CO)X¹(A)—

wherein:

X¹ is a heteroatom selected from the group consisting of O, S, N, and Se, usually one of the first three; and

A is a chain of at least 2 carbon atoms and usually not more than 6 carbon atoms substituted with an molecular tag, where preferably the other valences of A are satisfied by hydrogen, although the chain may be substituted with other groups, such as alkyl, aryl, heterocyclic groups, etc., A generally being not more than 10 carbon atoms.

Also of interest are heterocyclic compounds, such as diheterocyclopentadienes, as exemplified by substituted imidazoles, thiazoles, oxazoles, etc., where the rings will usually be substituted with at least one aromatic group and in some instances hydrolysis will be necessary to release the molecular tag.

Also of interest are tellurium (Te) derivatives, where the Te is bonded to an ethylene group having a hydrogen atom β to the Te atom, wherein the ethylene group is part of an alicyclic or heterocyclic ring, that may have an oxo group, preferably fused to an aromatic ring and the other valence of the Te is bonded to the molecular tag. The rings may be coumarin, benzoxazine, tetralin, etc.

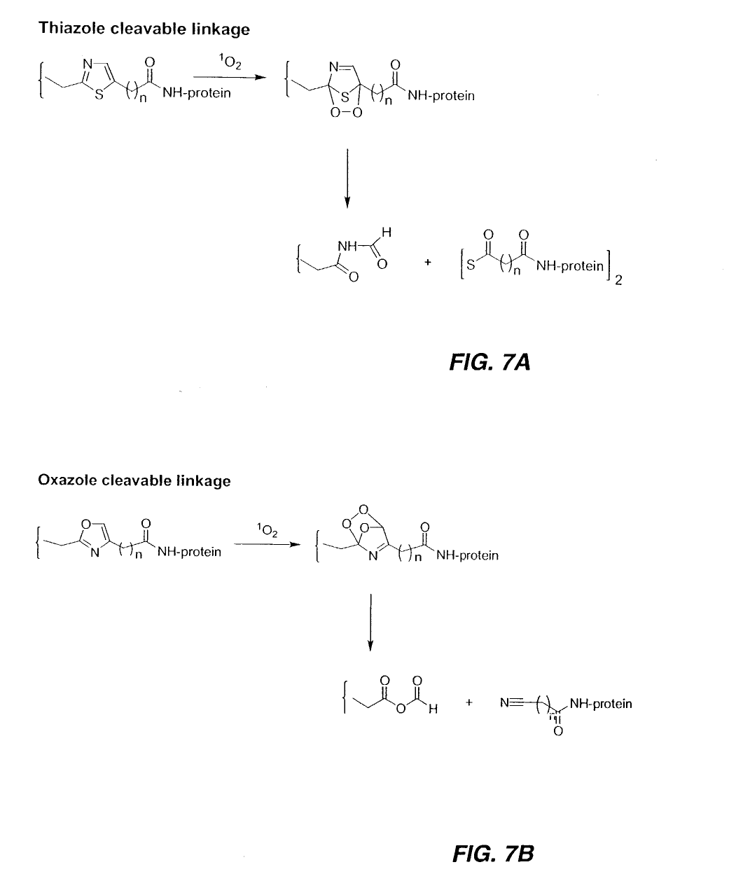

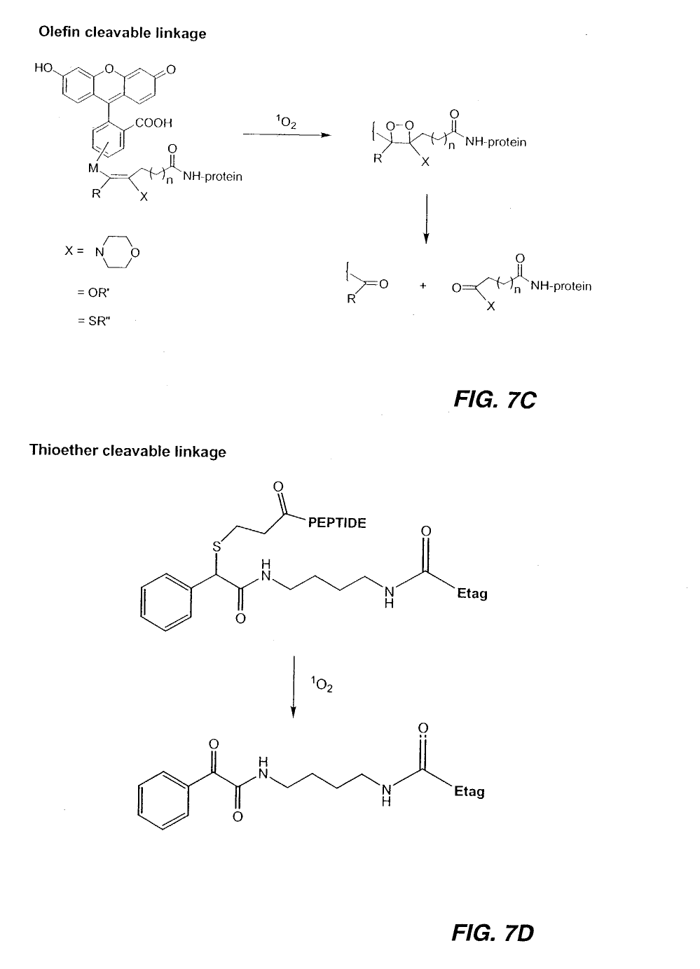

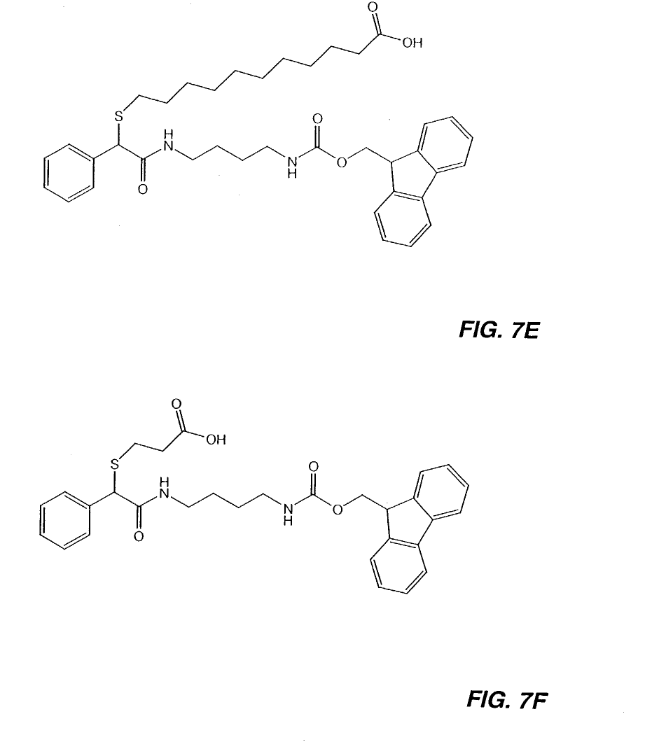

Several preferred cleavable linkages and their cleavage products are illustrated in FIGS. 7A-F. The thiazole cleavable linkage, “—CH₂-thiazole-(CH2)ₙ—C(═O)—NH—,” shown in FIG. 7A, results in an molecular tag with the moiety “—CH₂—C(═O)—NH—CHO.” Preferably, n is in the range of from 1 to 12, and more preferably, from 1 to 6. The oxazole cleavable linkage, “—CH₂-oxazole-(CH2)ₙ—C(═O)—NH—,” shown in FIG. 7B, results in an molecular tag with the moiety “13 CH₂—C(═O)O—CHO.” An olefin cleavable linkage is shown in FIG. 7C with D being a fluorescein dye. Cleavage of the illustrated olefin linkage results in an molecular tag of the form: “R—(C═O)—M—D,” where “R” may be any substituent within the general description of the molecular tags, E, provided above. Preferably, R is an electron-donating group, e.g. Ullman et al, U.S. Pat. No. 6,251,581; Smith and March, March's Advanced Organic Chemistry: Reactions, Mechanisms, and Structure, 5^{th} Edition (Wiley-Interscience, New York, 2001); and the like. More preferably, R is an electron-donating group having from 1-8 carbon atoms and from 0 to 4 heteroatoms selected from the group consisting of O, S, and N. In further preference, R is —N(Q)₂, —OQ, p-[C₆H₄N(Q)₂], furanyl, n-alkylpyrrolyl, 2-indolyl, or the like, where Q is alkyl or aryl. In further reference to the olefin cleavable linkage of FIG. 7C, substituents “X” and “R” are equivalent to substituents “X” and “Y” of the above formula describing cleavable linkage, L. In particular, X in FIG. 7C is preferably morpholino, —OR′, or —SR″, where R′ and R″ are aliphatic, aromatic, alicyclic or heterocyclic having from 1 to 8 carbon atoms and 0 to 4 heteroatoms selected from the group consisting of O, S, and N. A preferred thioether cleavable linkage is illustrated in FIG. 7D having the form “—(CH₂)₂—S—CH(C₆H₅)C(═O)NH—(CH₂)ₙ—NH—,” wherein n is in the range of from 2 to 12, and more preferably, in the range of from 2 to 6. Thioether cleavable linkages of the type shown in FIG. 7D may be formed by way of precursor compounds shown in FIGS. 7E and 7F. After reaction with the amino group and attachment, the Fmoc protection group is removed to produce a free amine which is then reacted with an NHS ester of the molecular tag, such as compounds produced by the schemes of FIGS. 1, 2, and 4, with the exception that the last reaction step is the addition of an NHS ester, instead of a phosphoramidite group.

Molecular tag, —(M,D), is a water soluble organic compound that is stable with respect to the active species, especially singlet oxygen, and that includes a detection or reporter group. Otherwise, E may vary widely in size and structure. In one aspect, E has a molecular weight in the range of from about 100 to about 2500 daltons, more preferably, from about 100 to about 1500 daltons. Preferred structures of —(M,D) are described more fully below. The detection group may generate an electrochemical, fluorescent, or chromogenic signal. Preferably, the detection group generates a fluorescent signal.

Molecular tags within a plurality of a composition each have either a unique chromatographic separation characteristics and/or a unique optical property with respect to the other members of the same plurality. In one aspect, the chromatographic separation characteristic is retention time in the column used for separation. In another aspect, the optical property is a fluorescence property, such as emission spectrum, fluorescence lifetime, fluorescence intensity at a given wavelength or band of wavelengths, or the like. Preferably, the fluorescence property is fluorescence intensity. For example, each molecular tag of a plurality may have the same fluorescent emission properties, but each will differ from one another by virtue of a unique retention time in the column of choice. On the other hand, or two or more of the molecular tags of a plurality may have identical retention times, but they will have unique fluorescent properties, e.g. spectrally resolvable emission spectra, so that all the members of the plurality are distinguishable by the combination of molecular separation and fluorescence measurement.

In one aspect, molecular tag is (M, D), where M is a mobility-modifying moiety and D is a detection moiety. The notation “(M, D)” is used to indicate that the ordering of the M and D moieties may be such that either moiety can be adjacent to the cleavable linkage, L. That is, “primer-L—(M, D)” designates binding compound of either of two forms: “primer-L—M—D” or “primer-L—D—M.”

Detection moiety, D, may be a fluorescent label or dye, a chromogenic label or dye, an electrochemical label, or the like. Preferably, D is a fluorescent dye. Exemplary fluorescent dyes for use with the invention include water-soluble rhodamine dyes, fluoresceins, 4,7-dichlorofluoresceins, benzoxanthene dyes, and energy transfer dyes, disclosed in the following references: Handbook of Molecular Probes and Research Reagents, 8^{th} ed., (Molecular Probes, Eugene, 2002); Lee et al, U.S. Pat. No. 6,191,278; Lee et al, U.S. Pat. No. 6,372,907; Menchen et al, U.S. Pat. No. 6,096,723; Lee et al, U.S. Pat. No. 5,945,526; Lee et al, Nucleic Acids Research, 25: 2816-2822 (1997); Hobb, Jr., U.S. Pat. No. 4,997,928; Khanna et al., U.S. Pat. No. 4,318,846; Reynolds, U.S. Pat. No. 3,932,415; Eckert et al, U.S. Pat. No. 2,153,059; Eckert et al, U.S. Pat. No. 2,242,572; Taing et al, International patent publication WO 02/30944; and the like. Further specific exemplary fluorescent dyes include 5- and 6-carboxyrhodamine 6G; 5- and 6-carboxy-X-rhodamine, 5- and 6-carboxytetramethylrhodamine, 5- and 6-carboxyfluorescein, 5- and 6-carboxy-4,7-dichlorofluorescein, 2′,7′-dimethoxy-5- and 6-carboxy-4,7-dichlorofluorescein, 2′,7′-dimethoxy-4′,5′-dichloro-5- and 6-carboxyfluorescein, 2′,7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy4,7-dichlorofluorescein, 2′,7′-dichloro-5- and 6-carboxy-4,7-dichlorofluorescein, and 2′,4′,5′,7′-tetrachloro-5- and 6-carboxy-4,7-dichlorofluorescein. Most preferably, D is a fluorescein or a fluorescein derivative.

The size and composition of mobility-modifying moiety, M, can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility-modifying moiety has from about 0 to about 40, more usually from about 0 to about 30 heteroatoms, which in addition to the heteroatoms indicated above may include halogen or other heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (includes ammonium), phosphonium, sulfonium, oxonium, etc., where substituents are generally aliphatic of from about 1-6 carbon atoms, the total number of carbon atoms per heteroatom, usually be less than about 12, usually less than about 9. The side chains include amines, ammonium salts, hydroxyl groups, including phenolic groups, carboxyl groups, esters, amides, phosphates, heterocycles. M may be a homo-oligomer or a hetero-oligomer, having different monomers of the same or different chemical characteristics, e.g., nucleotides and amino acids.

In another aspect, (M,D) moieties are constructed from chemical scaffolds used in the generation of combinatorial libraries. For example, the following references describe scaffold compound useful in generating diverse mobility modifying moieties: peptoids (PCT Publication No WO 91/19735, Dec. 26, 1991), encoded peptides (PCT Publication WO 93/20242, Oct. 14 1993), random bio-oligomers, (PCT Publication WO 92/00091, Jan. 9, 1992), benzodiazepines (U.S. Pat. No. 5,288,514), diverseness such as hydantoins, benzodiazepines and dipeptides (Hobbs DeWitt, S. et al., Proc. Nat. Acad. Sci. U.S.A. 90: 6909-6913 (1993), vinylogous polypeptides (Hagihara et al. J.Amer. Chem. Soc. 114: 6568 (1992)), nonpeptidal peptidomimetics with a Beta-D-Glucose scaffolding (Hirschmann, R. et al., J.Amer. Chem. Soc. 114: 9217-9218 (1992)), analogous organic syntheses of small compound libraries (Chen, C. et al. J.Amer. Chem. Soc. 116: 2661(1994)), oligocarbamates (Cho, C. Y. et al. Science 261: 1303(1993)), peptidyl phosphonates (Campbell, D. A. et al., J. Org. Chem. 59:658(1994)); Cheng et al, U.S. Pat. No. 6,245,937; Heizmann et al, “Xanthines as a scaffold for molecular diversity,” Mol. Divers. 2: 171-174 (1997); Pavia et al, Bioorg. Med. Chem., 4: 659-666 (1996); Ostresh et al, U.S. Pat. No. 5,856,107; Gordon, E. M. et al., J. Med. Chem. 37: 1385 (1994); and the like. Preferably, in this aspect, D is a substituent on a scaffold and M is the rest of the scaffold.

In yet another aspect, (M, D) moieties are constructed from one or more of the same or different common or commercially available linking, cross-linking, and labeling reagents that permit facile assembly, especially using a commercial DNA or peptide synthesizer for all or part of the synthesis. In this aspect, (M, D) moieties are made up of subunits usually connected by phosphodiester and amide bonds. Exemplary, precusors include, but are not limited to, dimethoxytrityl (DMT)-protected hexaethylene glycol phosphoramidite, 6-(4-Monomethoxytritylamino)hexyl-(2-cyanoethyl)-(N,N-diisopropyl)-phosphoramidite, 12-(4-Monomethoxytritylamino)dodecyl-(2-cyanoethyl)-(N,N-diisopropyl)-phosphoramidite, 2-[2-(4-Monomethoxytrityl)aminoethoxy]ethyl-(2-cyanoethyl), N,N-diisopropyl)-phosphoramidite, (S-Trityl-6-mercaptohexyl)-(2-cyanoethyl)-(N,N-diisopropyl)-phosphoramidite, 5′-Fluorescein phosphoramidite, 5′-Hexachloro-Fluorescein Phosphoramidite, 5′-Tetrachloro-Fluorescein Phosphoramidite, 9-O-Dimethoxytrityl-triethylene glycol,1-[(2-cyanoethyl)-(N,N-diisopropyl)]-phosphoramidite, 3(4,4′Dimethoxytrityloxy)propyl-1-[(2-cyanoethyl)-(N,N-diisopropyl)]-phosphoramidite, 5′-O-Dimethoxytrityl-1′,2′-Dideoxyribose-3′-[(2-cyanoethyl)-(N,N-diisopropyl)]-phosphoramidite, 18-O Dimethoxytritylhexaethyleneglycol, 1-[(2-cyanoethyl)-(N,N-diisopropyl)]-phosphoramidite, 12-(4,4′-Dimethoxytrityloxy)dodecyl-1-[(2-cyanoethyl)-(N,N-diisopropyl)]-phosphoramidite, 1,3-bis-[5-(4,4′-dimethoxytrityloxy)pentylamido]propyl-2-[(2-cyanoethyl)-(N,N-diisopropyl)]-phosphoramidite, 1-[5-(4,4′-dimethoxytrityloxy)pentylamido]-3-[5-fluorenomethoxycarbonyloxy pentylamido]-propyl-2-[(2-cyanoethyl)-(N,N-diisopropyl)]-phosphoramidite, Tris-2,2,2-[3-(4,4′-dimethoxytrityloxy)propyloxymethyl]ethyl-[(2-cyanoethyl)-(N,N-diisopropyl)]-phosphoramidite, succinimidyl trans-4-(maleimidylmethyl)cyclohexane-1-carboxylate (SMCC), succinimidyl 3-(2-pyridyldithio)propionate (SPDP), succinimidyl acetylthioacetate, Texas Red-X-succinimidyl ester, 5- and 6-carboxytetramethylrhodamine succinimidyl ester, bis-(4-carboxypiperidinyl)sulfonerhodamine di(succinimidyl ester), 5- and 6-((N-(5-aminopentyl)aminocarbonyl)tetramethylrhodamine, succinimidyl 4-(p-maleimidophenyl)butyrate (SMPB); N-γ-maleimidobutyryl-oxysuccinimide ester (GMBS); p-nitrophenyl iodoacetate (NPIA); 4-(4-N-maleimidophenyl)butyric acid hydrazide (MPBH); and like reagents. The above reagents are commercially available, e.g. from Glen Research (Sterling, Va.), Molecular Probes (Eugene, Oreg.), Pierce Chemical, and like reagent providers. Use of the above reagents in conventional synthetic schemes is well known in the art, e.g. Hermanson, Bioconjugate Techniques (Academic Press, New York, 1996). In particular, M may be constructed from the following reagents: dimethoxytrityl (DMT)-protected hexaethylene glycol phosphoramidite, 6-(4-Monomethoxytritylamino)hexyl-(2-cyanoethyl)-(N,N-diisopropyl)-phosphoramidite, 12-(4-Monomethoxytritylamino)dodecyl-(2-cyanoethyl)-(N,N-diisopropyl)-phosphoramidite, 2-[2-(4-Monomethoxytrityl)aminoethoxy]ethyl-(2-cyanoethyl), N,N-diisopropyl)-phosphoramidite, (S-Trityl-6-mercaptohexyl)-(2-cyanoethyl)-(N,N-diisopropyl)-phosphoramidite, 9-O-Dimethoxytrityl-triethylene glycol,1-[(2-cyanoethyl)-(N,N-diisopropyl)]-phosphoramidite, 3(4,4′Dimethoxytrityloxy)propyl-1-[(2-cyanoethyl)-(N,N-diisopropyl)]-phosphoramidite, 5′-O-Dimethoxytrityl-1′,2′-Dideoxyribose-3′-[(2-cyanoethyl)-(N,N-diisopropyl)]-phosphoramidite, 18-O Dimethoxytritylhexaethyleneglycol,1-[(2-cyanoethyl)-(N,N-diisopropyl)]-phosphoramidite, 12-(4,4′-Dimethoxytrityloxy)dodecyl-1-[(2-cyanoethyl)-(N,N-diisopropyl)]-phosphoramidite, 1,3-bis-[5-(4,4′-dimethoxytrityloxy)pentylamido]propyl-2-[(2-cyanoethyl)-(N,N-diisopropyl)]-phosphoraramide, 1-[5-(4,4′-dimethoxytrityloxy)pentylamido]-3-[5-fluorenomethoxycarbonyloxy pentylamido]-propyl-2-[(2-cyanoethyl)-(N,N-diisopropyl)]-phosphoramidite, Tris-2,2,2-[3-(4,4′-dimethoxytrityloxy)propyloxymethyl]ethyl-[(2-cyanoethyl)-(N,N-diisopropyl)]-phosphoramidite, succinimidyl trans-4-(maleimidylmethyl)cyclohexane-1-carboxylate (SMCC), succinimidyl 3-(2-pyridyldithio)propionate (SPDP), succinimidyl acetylthioacetate, succinimidyl 4-(p-maleimidophenyl)butyrate (SMPB); N-γ-maleimidobutyryl-oxysuccinimide ester (GMBS); p-nitrophenyl iodoacetate (NPIA); and 4-(4-N-maleimidophenyl)butyric acid hydrazide (MPBH).

M may also comprise polymer chains prepared by known polymer subunit synthesis methods. Methods of forming selected-length polyethylene oxide-containing chains are well known, e.g. Grossman et al, U.S. Pat. No. 5,777,096. It can be appreciated that these methods, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), polypeptides, oligosaccharides, polyurethanes, polyamides, polysulfonamides, polysulfoxides, polyphosphonates, and block copolymers thereof, including polymers composed of units of multiple subunits linked by charged or uncharged linking groups. In addition to homopolymers, the polymer chains used in accordance with the invention include selected-length copolymers, e.g., copolymers of polyethylene oxide units alternating with polypropylene units. As another example, polypeptides of selected lengths and amino acid composition (i.e., containing naturally occurring or man-made amino acid residues), as homopolymers or mixed polymers.

### Cleavage-Inducing Moieties Producing Active Species

A cleavage-inducing moiety is a group that produces an active species that is capable of cleaving a cleavable linkage, preferably by oxidation. Preferably, the active species is a chemical species that exhibits short-lived activity so that its cleavage-inducing effects are only in the proximity of the site of its generation. Either the active species is inherently short lived, so that it will not create significant background because beyond the proximity of its creation, or a scavenger is employed that efficiently scavenges the active species, so that it is not available to react with cleavable linkages beyond a short distance from the site of its generation. Illustrative active species include singlet oxygen, hydrogen peroxide, NADH, and hydroxyl radicals, phenoxy radical, superoxide, and the like. Illustrative quenchers for active species that cause oxidation include polyenes, carotenoids, vitamin E, vitamin C, amino acid-pyrrole N-conjugates of tyrosine, histidine, and glutathione, and the like, e.g. Beutner et al, Meth. Enzymol., 319: 226-241 (2000).

An important consideration for the cleavage-inducing moiety and the cleavable linkage is that they not be so far removed from one another when bound to a target protein that the active species generated by the sensitizer diffuses and loses its activity before it can interact with the cleavable linkage. Accordingly, a cleavable linkage preferably are within 1000 nm, preferably 20-100 nm of a bound cleavage-inducing moiety. This effective range of a cleavage-inducing moiety is referred to herein as its “effective proximity.”

Generators of active species include enzymes, such as oxidases, such as glucose oxidase, xanthene oxidase, D-amino acid oxidase, NADH-FMN oxidoreductase, galactose oxidase, glyceryl phosphate oxidase, sarcosine oxidase, choline oxidase and alcohol oxidase, that produce hydrogen peroxide, horse radish peroxidase, that produces hydroxyl radical, various dehydrogenases that produce NADH or NADPH, urease that produces ammonia to create a high local pH.

A sensitizer is a compound that can be induced to generate a reactive intermediate, or species, usually singlet oxygen. Preferably, a sensitizer used in accordance with the invention is a photosensitizer. Other sensitizers included within the scope of the invention are compounds that on excitation by heat, light, ionizing radiation, or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed in the following references: Di Mascio et al, FEBS Lett., 355: 287 (1994)(peroxidases and oxygenases); Kanofsky, J.Biol. Chem. 258: 5991-5993 (1983)(lactoperoxidase); Pierlot et al, Meth. Enzymol., 319: 3-20 (2000)(thermal lysis of endoperoxides); and the like.

Attachment of a binding agent to the cleavage-inducing moiety may be direct or indirect, covalent or non-covalent and can be accomplished by well-known techniques, commonly available in the literature. See, for example, “Immobilized Enzymes,” Ichiro Chibata, Halsted Press, New York (1978); Cuatrecasas, J. Biol. Chem., 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group to a binding agent may vary widely, depending upon the nature of the compound being linked, the effect of the distance on the specific binding properties and the like.

It may be desirable to have multiple cleavage-inducing moieties attached to a binding agent to increase, for example, the number of active species generated. This can be accomplished with a polyfunctional material, normally polymeric, having a plurality of functional groups, e.g., hydroxy, amino, mercapto, carboxy, ethylenic, aldehyde, etc., as sites for linking. Alternatively a support may be used. The support can have any of a number of shapes, such as particle including bead, film, membrane, tube, well, strip, rod, and the like. For supports in which photosensitizer is incorporated, the surface of the support is, preferably, hydrophilic or capable of being rendered hydrophilic and the body of the support is, preferably, hydrophobic. The support may be suspendable in the medium in which it is employed. Examples of suspendable supports, by way of illustration and not limitation, are polymeric materials such as latex, lipid bilayers, oil droplets, cells and hydrogels. Other support compositions include glass, metals, polymers, such as nitrocellulose, cellulose acetate, poly(vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials. Attachment of binding agents to the support may be direct or indirect, covalent or non-covalent and can be accomplished by well-known techniques, commonly available in the literature as discussed above. See, for example, “Immobilized Enzymes,” Ichiro Chibata, supra. The surface of the support will usually be polyfunctional or be capable of being polyfunctionalized or be capable of binding to a target-binding moiety, or the like, through covalent or specific or non-specific non-covalent interactions.

The cleavage-inducing moiety may be associated with the support by being covalently or non-covalently attached to the surface of the support or incorporated into the body of the support. Linking to the surface may be accomplished as discussed above. The cleavage-inducing moiety may be incorporated into the body of the support either during or after the preparation of the support. In general, the cleavage-inducing moiety is associated with the support in an amount necessary to achieve the necessary amount of active species. Generally, the amount of cleavage-inducing moiety is determined empirically.

### Photosensitizers as Cleavage-Inducing Moieties

As mentioned above, the preferred cleavage-inducing moiety in accordance with the present invention is a photosensitizer that produces singlet oxygen. As used herein, “photosensitizer” refers to a light-adsorbing molecule that when activated by light converts molecular oxygen into singlet oxygen. Guidance for selecting, forming conjugates from, using, and synthesizing photosensitizers is available in the literature, e.g. in the fields of photodynamic therapy, immunodiagnostics, and the like. The following are exemplary references: Ullman, et al., Proc. Natl. Acad. Sci. USA 91, 5426-5430 (1994); Strong et al, Ann. New York Acad. Sci., 745: 297-320 (1994); Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Wasserman and R. W. Murray. Singlet Oxygen. (Academic Press, New York, 1979); Baumstark, Singlet Oxygen, Vol. 2 (CRC Press Inc., Boca Raton, Fla. 1983); and Turro, Modern Molecular Photochemistry (University Science Books, 1991).

The photosensitizers are sensitizers for generation of singlet oxygen by excitation with light. The photosensitizers include dyes and aromatic compounds, and are usually compounds comprised of covalently bonded atoms, usually with multiple conjugated double or triple bonds. The compounds typically absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹ cm⁻¹, preferably, about 5,000 M⁻¹ cm⁻¹, more preferably, about 50,000 M⁻¹ cm⁻¹, at the excitation wavelength. The lifetime of an excited state produced following absorption of light in the absence of oxygen will usually be at least about 100 nanoseconds, preferably, at least about 1 millisecond. In general, the lifetime must be sufficiently long to permit cleavage of a linkage in a reagent in accordance with the present invention. Such a reagent is normally present at concentrations as discussed below. The photosensitizer excited state usually has a different spin quantum number (S) than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures.

A large variety of light sources are available to photo-activate photosensitizers to generate singlet oxygen. Both polychromatic and monchromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; and the like.

Examples of photosensitizers that may be utilized in the present invention are those that have the above properties and are enumerated in the following references: Turro, Modern Molecular Photochemistry (cited above); Singh and Ullman, U.S. Pat. No. 5,536,834; Li et al, U.S. Pat. No. 5,763,602; Ulhman, et al., Proc. Natl. Acad. Sci. USA 91,5426-5430 (1994); Strong et al, Ann. New York Acad. Sci., 745: 297-320 (1994); Martin et al, Methods Enzymol., 186: 635-645 (1990);Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Wohrle, Chimia, 45: 307-310 (1991); Thetford, European patent publ. 0484027; Sessler et al, SPIE, 1426: 318-329 (1991); Madison et al, Brain Research, 522: 90-98 (1990); Polo et al, Inorganica Chimica Acta, 192: 1-3 (1992); Demas et al, J. Macromol. Sci., A25: 1189-1214 (1988); and the like. Exemplary photosensitizers are listed in Table 1b.

TABLE 1b
Exemplary Photosensitizers

| | |
|---|---|
| Hypocrellin A | Tetraphenylporphyrin |
| Hypocrellin B | Halogenated derivatives |
| | of rhodamine dyes |
| Hypericin | metallo-Porphyrins |
| Halogenated derivatives | Phthalocyanines |
| of fluorescein dyes | |
| Rose bengal | Naphthalocyanines |
| Merocyanine 540 | Texaphyrin-type macrocycles |
| Methylene blue | Hematophorphyrin |
| 9-Thioxanthone | 9,10-Dibromoanthracene |
| Chlorophylls | Benzophenone |
| Phenaleone | Chlorin e6 |
| Protoporphyrin | Perylene |
| Benzoporphryin A monacid | Benzoporphryin B monacid |

In certain embodiments the photosensitizer moiety comprises a support, as discussed above with respect to the cleavage-inducing moiety. The photosensitizer may be associated with the support by being covalently or non-covalently attached to the surface of the support or incorporated into the body of the support as discussed above. In general, the photosensitizer is associated with the support in an amount necessary to achieve the necessary amount of singlet oxygen. Generally, the amount of photosensitizer is determined empirically. Photosensitizers used as the photosensitizer are preferably relatively non-polar to assure dissolution into a lipophilic member when the photosensitizer is incorporated in, for example, a latex particle to form photosensitizer beads, e.g. as disclosed by Pease et al., U.S. Pat. No. 5,709,994. For example, the photosensitizer rose bengal is covalently attached to 0.5 micron latex beads by means of chloromethyl groups on the latex to provide an ester linking group, as described in J. Amer. Chem. Soc., 97: 3741 (1975).

Conjugation of sensitizer molecules to assay reagents: Sensitizer molecules can be conjugated to other molecules, e.g. oligonucleotides, by various methods and in various configurations. For example, an activated (NHS ester, aldehyde, sulfonyl chloride, etc) sensitizer (Rose Bengal, phthalocyanine, etc.) can be reacted with reactive amino-group containing moieties (antibody, avidin or other proteins, H₂N-LC-Biotin, aminodextran, amino-group containing other small and large molecules). Such conjugates can be used directly (for example the antibody-sensitizer conjugate, Biotin-LC-sensitizer, etc.) in various assays.

### Separation of Released Molecular Tags

As mentioned above, molecular tags are designed for separation by a separation technique that can distinguish molecular tags based on one or more physical, chemical, and/or optical characteristics. Preferably, such separation technique is capable of providing quantitative information as well as qualitative information about the presence or absence of molecular tags (and therefore, corresponding analytes). In one aspect, a liquid phase separation technique is employed so that a solution, e.g. buffer solution, reaction solvent, or the like, containing a mixture of molecular tags is processed to bring about separation of individual kinds of molecular tags. Usually, such separation is accompanied by the differential movement of molecular tags from such a starting mixture along a path until discernable peaks or bands form that correspond to regions of increased concentration of the respective molecular tags. Such a path may be defined by a fluid flow, electric field, magnetic field, or the like. The selection of a particular separation technique depends on several factors including the expense and convenience of using the technique, the resolving power of the technique given the chemical nature of the molecular tags, the number of molecular tags to be separated, the type of detection mode employed, and the like. Preferably, molecular tags are electrophoretically or chromatographically separated.

A. Electrophoretic Separation

Methods for electrophoresis of are well known and there is abundant guidance for one of ordinary skill in the art to make design choices for forming and separating particular pluralities of molecular tags. The following are exemplary references on electrophoresis: Krylov et al, Anal. Chem., 72: 111R-128R (2000); P. D. Grossman and J. C. Colburn, Capillary Electrophoresis: Theory and Practice, Academic Press, Inc., NY (1992); U.S. Pat. Nos. 5,374,527; 5,624,800; 5,552,028; ABI PRISM 377 DNA Sequencer User's Manual, Rev. A, January 1995, Chapter 2 (Applied Biosystems, Foster City, Calif.); and the like. In one aspect, molecular tags are separated by capillary electrophoresis. Design choices within the purview of those of ordinary skill include but are not limited to selection of instrumentation from several commercially available models, selection of operating conditions including separation media type and concentration, pH, desired separation time, temperature, voltage, capillary type and dimensions, detection mode, the number of molecular tags to be separated, and the like.

In one aspect of the invention, during or after electrophoretic separation, the molecular tags are detected or identified by recording fluorescence signals and migration times (or migration distances) of the separated compounds, or by constructing a chart of relative fluorescent and order of migration of the molecular tags (e.g., as an electropherogram). To perform such detection, the molecular tags can be illuminated by standard means, e.g. a high intensity mercury vapor lamp, a laser, or the like. Typically, the molecular tags are illuminated by laser light generated by a He—Ne gas laser or a solid-state diode laser. The fluorescence signals can then be detected by a light-sensitive detector, e.g., a photomultiplier tube, a charged-coupled device, or the like. Exemplary electrophoresis detection systems are described elsewhere, e.g., U.S. Pat. Nos. 5,543,026; 5,274,240; 4,879,012; 5,091,652; 6,142,162; or the like. In another aspect, molecular tags may be detected electrochemically detected, e.g. as described in U.S. Pat. No. 6,045,676.

Electrophoretic separation involves the migration and separation of molecules in an electric field based on differences in mobility. Various forms of electrophoretic separation include, by way of example and not limitation, free zone electrophoresis, gel electrophoresis, isoelectric focusing, isotachophoresis, capillary electrochromatography, and micellar electrokinetic chromatography. Capillary electrophoresis involves electroseparation, preferably by electrokinetic flow, including electrophoretic, dielectrophoretic and/or electroosmotic flow, conducted in a tube or channel of from about 1 to about 200 micrometers, usually, from about 10 to about 100 micrometers cross-sectional dimensions. The capillary may be a long independent capillary tube or a channel in a wafer or film comprised of silicon, quartz, glass or plastic.

In capillary electroseparation, an aliquot of the reaction mixture containing the molecular tags is subjected to electroseparation by introducing the aliquot into an electroseparation channel that may be part of, or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents used in the present invention and/or the nature of the cleaved labels, the nature of the reaction medium and so forth. The parameters for the electroseparation including those for the medium and the electric potential are usually optimized to achieve maximum separation of the desired components. This may be achieved empirically and is well within the purview of the skilled artisan.

Detection may be by any of the known methods associated with the analysis of capillary electrophoresis columns including the methods shown in U.S. Pat. Nos. 5,560,811 (column 11, lines 19-30), 4,675,300, 4,274,240 and 5,324,401, the relevant disclosures of which are incorporated herein by reference. Those skilled in the electrophoresis arts will recognize a wide range of electric potentials or field strengths may be used, for example, fields of 10 to 1000 V/cm are used with about 200 to about 600 V/cm being more typical. The upper voltage limit for commercial systems is about 30 kV, with a capillary length of about 40 to about 60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from about 180 to about 1500 nm, usually about 220 to about 800 nm, more usually about 450 to about 700 nm, to have low transmission losses. Suitable materials include fused silica, plastics, quartz, glass, and so forth.

B. Chromatographic Separation

In one aspect of the invention, pluralities of molecular tags are designed for separation by chromatography based on one or more physical characteristics that include but are not limited to molecular weight, shape, solubility, pKa, hydrophobicity, charge, polarity, or the like. A chromatographic separation technique is selected based on parameters such as column type, solid phase, mobile phase, and the like, followed by selection of a plurality of molecular tags that may be separated to form distinct peaks or bands in a single operation. Several factors determine which HPLC technique is selected for use in the invention, including the number of molecular tags to be detected (i.e. the size of the plurality), the estimated quantities of each molecular tag that will be generated in the assays, the availability and ease of synthesizing molecular tags that are candidates for a set to be used in multiplexed assays, the detection modality employed, and the availability, robustness, cost, and ease of operation of HPLC instrumentation, columns, and solvents. Generally, columns and techniques are favored that are suitable for analyzing limited amounts of sample and that provide the highest resolution separations. Guidance for making such selections can be found in the literature, e.g. Snyder et al, Practical HPLC Method Development, (John Wiley & Sons, New York, 1988); Millner, “High Resolution Chromatography: A Practical Approach”, Oxford University Press, New York (1999), Chi-San Wu, “Column Handbook for Size Exclusion Chromatography”, Academic Press, San Diego (1999), and Oliver, “HPLC of Macromolecules: A Practical Approach, Oxford University Press”, Oxford, England (1989). In particular, procedures are available for systematic development and optimization of chromatographic separations given conditions, such as column type, solid phase, and the like, e.g. Haber et al, J. Chromatogr. Sci., 38: 386-392 (2000); Outinen et al, Eur. J. Pharm. Sci., 6: 197-205 (1998); Lewis et al, J. Chromatogr., 592: 183-195 and 197-208 (1992); and the like.

In one aspect, initial selections of molecular tag candidates are governed by the physiochemical properties of molecules typically separated by the selected column and stationary phase. The initial selections are then improved empirically by following conventional optimization procedure, as described in the above reference, and by substituting more suitable candidate molecular tags for the separation objectives of a particular embodiment. In one aspect, separation objectives of the invention include (i) separation of the molecular tags of a plurality into distinguishable peaks or bands in a separation time of less than 60 minutes, and more preferably in less than 40 minutes, and still more preferably in a range of between 10 to 40 minutes, (ii) the formation of peaks or bands such that any pair has a resolution of at least 1.0, more preferably at least 1.25, and still more preferably, at least 1.50, (iii) column pressure during separation of less than 150 bar, (iv) separation temperature in the range of from 25° C. to 90° C., preferably in the range of from 35° C. to 80° C., and (v) the plurality of distinguishable peaks is in the range of from 5 to 30 and all of the peaks in the same chromatogram. As used herein, “resolution” in reference to two peaks or bands is the distance between the two peak or band centers divided by the average base width of the peaks, e.g. Snyder et al (cited above).

A chromatographic method is used to separate molecular tags based on their chromatographic properties. A chromatographic property can be, for example, a retention time of a molecular tag on a specific chromatographic medium under defined conditions, or a specific condition under which a molecular tag is eluted from a specific chromatographic medium. A chromatographic property of a molecular tag can also be an order of, elution, or pattern of elution, of a molecular tag contained in a group or set of molecular tags being chromatographically separated using a specific chromatographic medium under defined conditions. A chromatographic property of a molecular tag is determined by the physical properties of the molecular tag and its interactions with a chromatographic medium and mobile phase. Defined conditions for chromatography include particular mobile phase solutions, column geometry, including column diameter and length, pH, flow rate, pressure and temperature of column operation, and other parameters that can be varied to obtain the desired separation of molecular tags. A molecular tag, or chromatographic property of a molecular tag, can be detected using a variety of chromatography methods.

Sets of molecular tags detected in a single experiment generally are a group of chemically related molecules that differ by mass, charge, mass-charge ratio, detectable tag, such as differing fluorophores or isotopic labels, or other unique characteristic. Therefore, both the chemical nature of the molecular tag and the particular differences among molecular tags in a group of molecular tags can be considered when selecting a suitable chromatographic medium for separating molecular tags in a sample.

Separation of molecular tags by liquid chromatography can be based on physical characteristics of molecular tags such as charge, size and hydrophobicity of molecular tags, or functional characteristics such as the ability of molecular tags to bind to molecules such as dyes, lectins, drugs, peptides and other ligands on an affinity matrix. A wide variety of chromatographic media are suitable for separation of molecular tag based on charge, size, hydrophobicity and other chromatographic properties of molecular tags. Selection of a particular chromatographic medium will depend upon the properties of molecular tags employed.

Separated molecular tags can be detected using a variety of analytical methods, including detection of intrinsic properties of molecular tags, such as absorbance, fluorescence or electrochemical properties, as well as detection of a detection group or moiety attached to a molecular tag. Although not required, a variety of detection groups or moieties can be attached to molecular tags to facilitate detection after chromatographic separation.

Detection methods for use with liquid chromatography are well known, commercially available, and adaptable to automated and high-throughput sampling. The detection method selected for analysis of molecular tags will depend upon whether the molecular tags contain a detectable group or moiety, the type of detectable group used, and the physicochemical properties of the molecular tag and detectable group, if used. Detection methods based on fluorescence, electrolytic conductivity, refractive index, and evaporative light scattering can be used to detect various types of molecular tags.

A variety of optical detectors can be used to detect a molecular tag separated by liquid chromatography. Methods for detecting nucleic acids, polypeptides, peptides, and other macromolecules and small molecules using ultraviolet (UV)/visible spectroscopic detectors are well known, making UV/visible detection the most widely used detection method for HPLC analysis. Infrared spectrophotometers also can be used to detect macromolecules and small molecules when used with a mobile phase that is a transparent polar liquid.

Variable wavelength and diode-array detectors represent two commercially available types of UV/visible spectrophotometers. A useful feature of some variable wavelength UV detectors is the ability to perform spectroscopic scanning and precise absorbance readings at a variety of wavelengths while the peak is passing through the flowcell. Diode array technology provides the additional advantage of allowing absorbance measurements at two or more wavelengths, which permits the calculation of ratios of such absorbance measurements. Such absorbance rationing at multiple wavelengths is particularly helpful in determining whether a peak represents one or more than one molecular tag.

Fluorescence detectors can also be used to detect fluorescent molecular tags, such as those containing a fluorescent detection group and those that are intrinsically fluorescent. Typically, fluorescence sensitivity is relatively high, providing an advantage over other spectroscopic detection methods when molecular tags contain a fluorophore. Although molecular tags can have detectable intrinsic fluorescence, when a molecular tag contains a suitable fluorescent detection group, it can be possible to detect a single molecular tag in a sample.

Electrochemical detection methods are also useful for detecting molecular tags separated by HPLC. Electrochemical detection is based on the measurement of current resulting from oxidation or reduction reaction of the molecular tags at a suitable electrode. Since the level of current is directly proportional to molecular tag concentration, electrochemical detection can be used quantitatively, if desired.

Evaporative light scattering detection is based on the ability of particles to cause photon scattering when they traverse the path of a polychromatic beam of light. The liquid effluent from an HPLC is first nebulized and the resultant aerosol mist, containing the molecular tags, is directed through a light beam. A signal is generated that is proportional to the amount of the molecular tag present in a sample, and is independent of the presence or absence of detectable groups such as chromophores, fluorophores or electroactive groups. Therefore, the presence of a detection group or moiety on a molecular tag is not required for evaporative light scattering detection.

Mass spectrometry methods also can be used to detect molecular tags separated by HPLC. Mass spectrometers can resolve ions with small mass differences and measure the mass of ions with a high degree of accuracy and sensitivity. Mass spectrometry methods are well known in the art (see Burlingame et al. Anal. Chem. 70:647R-716R (1998); Kinter and Sherman, Protein Sequencing and Identification Using Tandem Mass Spectrometry Wiley-Interscience, New York (2000)).

Analysis of data obtained using any detection method, such as spectral deconvolution and quantitative analysis can be manual or computer-assisted, and can be performed using automated methods. A variety of computer programs can be used to determine peak integration, peak area, height and retention time. Such computer programs can be used for convenience to determine the presence of a molecular tag qualitatively or quantitatively. Computer programs for use with HPLC and corresponding detectors are well known to those skilled in the art and generally are provided with commercially available HPLC and detector systems.

A variety of commercially available systems are well-suited for high throughput analysis of molecular tags. Those skilled in the art can determine appropriate equipment, such as automated sample preparation systems and autoinjection systems, useful for automating HPLC analysis of molecular tags. Automated methods can be used for high-throughput analysis of molecular tags, for example, when a large number of samples are being processes or for multiplexed application of the methods of the invention for detecting target analytes. An exemplary HPLC instrumentation system suitable for use with the present invention is the Agilent 1100 Series HPLC system (Agilent Technologies, Palo Alto, Calif.).

Those skilled in the art will be aware of quality control measures useful for obtaining reliable analysis of molecular tags, particular when analysis is performed in a high-throughput format. Such quality control measures include the use of external and internal reference standards, analysis of chromatograph peak shape, assessment of instrument performance, validation of the experimental method, for example, by determining a range of linearity, recovery of sample, solution stability of sample, and accuracy of measurement.

### Synthesis of Molecular Tags and Binding Compounds

The chemistry for performing the types of syntheses to form the charge-imparting moiety or mobility modifier as a peptide chain is well known in the art. See, for example, Marglin, et al., Ann. Rev. Biochem. (1970) 39:841-866. In general, such syntheses involve blocking, with an appropriate protecting group, those functional groups that are not to be involved in the reaction. The free functional groups are then reacted to form the desired linkages. The peptide can be produced on a resin as in the Merrifield synthesis (Merrifield, J. Am. Chem. Soc. (1980) 85:2149-2154 and Houghten et al., Int. J. Pep. Prot. Res. (1980) 16:311-320. The peptide is then removed from the resin according to known techniques.

A summary of the many techniques available for the synthesis of peptides may be found in J. M. Stewart, et al., “Solid Phase Peptide Synthesis, W. H. Freeman Co, San Francisco (1969); and J. Meienhofer, “Hormonal Proteins and Peptides”, (1973), vol. 2, p. 46, Academic Press (New York), for solid phase peptide synthesis; and E. Schroder, et al., “The Peptides”, vol. 1, Academic Press (New York), 1965 for solution synthesis.

In general, these methods comprise the sequential addition of one or more amino acids, or suitably protected amino acids, to a growing peptide chain. Normally, a suitable protecting group protects either the amino or carboxyl group of the first amino acid. The protected or derivatized amino acid can then be either attached to an inert solid support or utilized in solution by adding the next amino acid in the sequence having the complementary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide. The protecting groups are removed, as desired, according to known methods depending on the particular protecting group utilized. For example, the protecting group may be removed by reduction with hydrogen and palladium on charcoal, sodium in liquid ammonia, etc.; hydrolysis with trifluoroacetic acid, hydrofluoric acid, and the like.

For synthesis of binding compounds employing phosphoramidite, or related, chemistry many guides are available in the literature: Handbook of Molecular Probes and Research Products, 8^{th} edition (Molecular Probes, Inc., Eugene, Oreg., 2002); Beaucage and Iyer, Tetrahedron, 48: 2223-2311 (1992); Molko et al, U.S. Pat. No. 4,980,460; Koster et al, U.S. Pat. No. 4,725,677; Caruthers et al, U.S. Pat. Nos. 4,415,732; 4,458,066; and 4,973,679; and the like. Many of these chemistries allow components of the binding compound to be conveniently synthesized on an automated DNA synthesizer, e.g. an Applied Biosystems, Inc. (Foster City, Calif.) model 392 or 394 DNA/RNA Synthesizer, or the like.

Synthesis of molecular tag reagents comprising nucleotides as part of the mobility-modifying moiety can be easily and effectively achieved via assembly on a solid phase support using standard phosphoramidite chemistries. The resulting mobility modifying moiety may be linked to the label and/or polypeptide-binding moiety as discussed above.

### Exemplary Synthetic Approaches for Molecular Tags

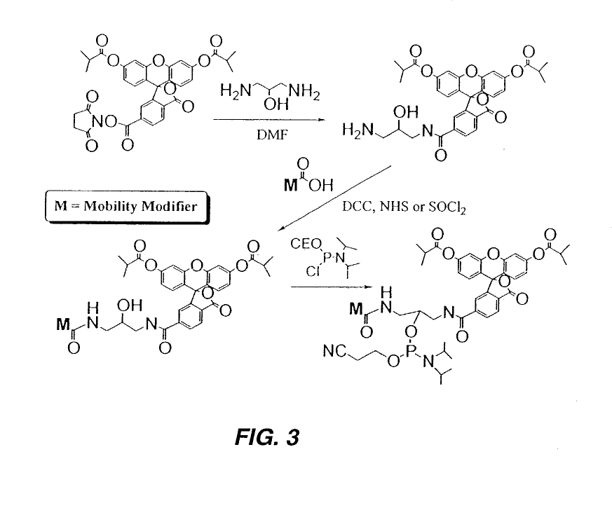

One exemplary synthetic approach is outlined in FIG. 1. Starting with commercially available 6-carboxy fluorescein, the phenolic hydroxyl groups are protected using an anhydride. Isobutyric anhydride in pyridine was employed but other variants are equally suitable. It is important to note the significance of choosing an ester functionality as the protecting group. This species remains intact throughout the phosphoramidite monomer synthesis as well as during oligonucleotide construction. These groups are not removed until the synthesized oligonucleotide is deprotected using ammonia. After protection the crude material is then activated in situ via formation of an N-hydroxysuccinimide ester (NHS-ester) using DCC as a coupling agent. The DCU by product is filtered away and an amino alcohol is added. Many amino alcohols are commercially available some of which are derived from reduction of amino acids. When the amino alcohol is of the form “H₂N—(CH₂)ₙ—OH,” n is in the range of from 2 to 12, and more preferably, from 2 to 6. Only the amine is reactive enough to displace N-hydroxysuccinimide. Upon standard extractive workup, a 95% yield of product is obtained. This material is phosphitylated to generate the phosphoramidite monomer. For the synthesis of additional molecular tags, a symmetrical bis-amino alcohol linker is used as the amino alcohol (FIG. 2). As such, the second amine is then coupled with a multitude of carboxylic acid derivatives (exemplified by several possible benzoic acid derivatives shown in FIG. 3 prior to the phosphitylation reaction.

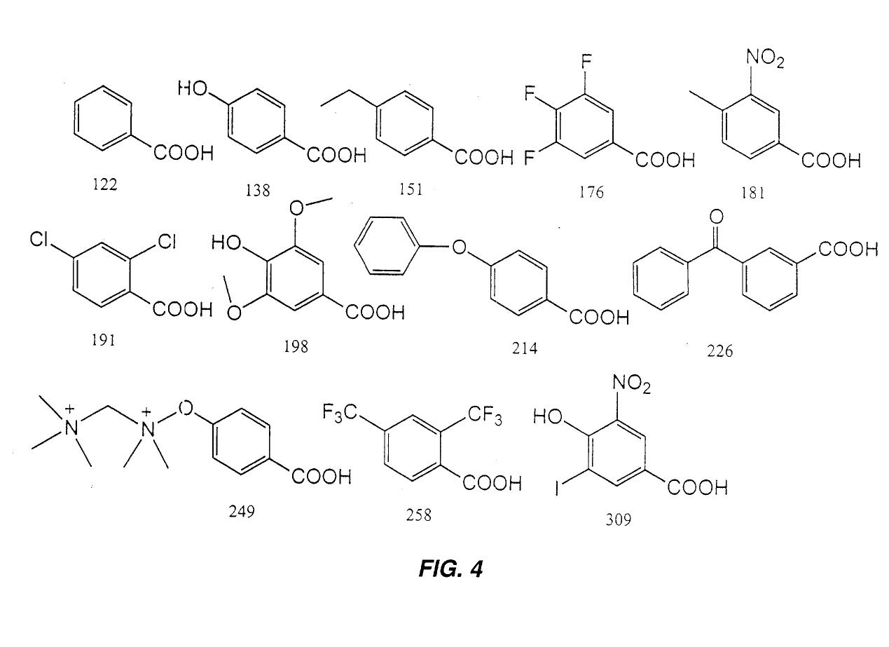

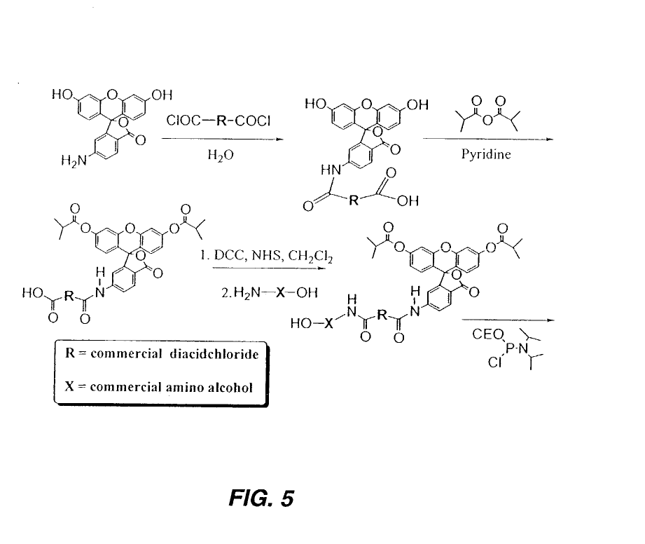

Alternatively, molecular tags may be made by an alternative strategy that uses 5-aminofluorescein as starting material (FIG. 4). Addition of 5-aminofluorescein to a great excess of a diacid dichloride in a large volume of solvent allows for the predominant formation of the monoacylated product over dimer formation. The phenolic groups are not reactive under these conditions. Aqueous workup converts the terminal acid chloride to a carboxylic acid. This product is analogous to 6-carboxyfluorescein, and using the same series of steps is converted to its protected phosphoramidite monomer. There are many commercially available diacid dichlorides and diacids, which can be converted to diacid dichlorides using SOCl₂ or acetyl chloride. There are many commercial diacid dichlorides and amino alcohols (FIG. 5). These synthetic approaches are ideally suited for combinatorial chemistry.

The molecular tags constructed with the schemes of FIGS. 1, 2, and 4 are further reacted either before or after phosphitylation to attach a cleavable linkage, e.g. using chemistry as described below.

The molecular tag may be assembled having an appropriate functionality at one end for linking to the polypeptide-binding moieties. A variety of functionalities can be employed. Thus, the functionalities normally present in a peptide, such as carboxy, amino, hydroxy and thiol may be the targets of a reactive functionality for forming a covalent bond. The molecular tag is linked in accordance with the chemistry of the linking group and the availability of functionalities on the polypeptide-binding moiety. For example, as discussed above for antibodies, and fragments thereof such as Fab′ fragments, specific for a polypeptide, a thiol group will be available for using an active olefin, e.g., maleimide, for thioether formation. Where lysines are available, one may use activated esters capable of reacting in water, such as nitrophenyl esters or pentafluorophenyl esters, or mixed anhydrides as with carbodiimide and half-ester carbonic acid. There is ample chemistry for conjugation in the literature, so that for each specific situation, there is ample precedent in the literature for the conjugation.

In an illustrative synthesis a diol is employed. Examples of such diols include an alkylene diol, polyalkylene diol, with alkylene of from 2 to 3 carbon atoms, alkylene amine or poly(alkylene amine) diol, where the alkylenes are of from 2 to 3 carbon atoms and the nitrogens are substituted, for example, with blocking groups or alkyl groups of from 1-6 carbon atoms, where one diol is blocked with a conventional protecting group, such as a dimethyltrityl group. This group can serve as the mass-modifying region and with the amino groups as the charge-modifying region as well. If desired, the mass modifier can be assembled by using building blocks that are joined through phosphoramidite chemistry. In this way the charge modifier can be interspersed between the mass modifier. For example, a series of polyethylene oxide molecules having 1, 2, 3, n units may be prepared. To introduce a number of negative charges, a small polyethylene oxide unit may be employed. The mass and charge-modifying region may be built up by having a plurality of the polyethylene oxide units joined by phosphate units. Alternatively, by employing a large spacer, fewer phosphate groups would be present, so that without large mass differences, large differences in mass-to-charge ratios may be realized.

The chemistry that is employed is the conventional chemistry used in oligonucleotide synthesis, where building blocks other than nucleotides are used, but the reaction is the conventional phosphoramidite chemistry and the blocking group is the conventional dimethoxytrityl group. Of course, other chemistries compatible with automated synthesizers can also be used. However, it is desirable to minimize the complexity of the process.

As mentioned above, in one embodiment the hub nucleus is a hydrophilic polymer, generally, an addition or condensation polymer with multiple functionality to permit the attachment of multiple moieties. One class of polymers that is useful for the reagents of the present invention comprises the polysaccharide polymers such as dextrans, sepharose, polyribose, polyxylose, and the like. For example, the hub may be dextran to which multiple molecular tags may be attached in a cleavable manner consistent with the present invention. A few of the aldehyde moieties of the dextran remain and may be used to attach the dextran molecules to amine groups on an oligonucleotide by reductive amination. In another example using dextran as the hub nucleus, the dextran may be capped with succinic anhydride and the resulting material may be linked to amine-containing oligonucleotides by means of amide formation.

Besides the nature of the linker and mobility-modifying moiety, as already indicated, diversity can be achieved by the chemical and optical characteristics of the fluorescer, the use of energy transfer complexes, variation in the chemical nature of the linker, which affects mobility, such as folding, interaction with the solvent and ions in the solvent, and the like. As already suggested, in one embodiment the linker is an oligomer, where the linker may be synthesized on a support or produced by cloning or expression in an appropriate host. Conveniently, polypeptides can be produced where there is only one cysteine or serine/threonine/tyrosine, aspartic/glutamic acid, or lysine/arginine/histidine, other than an end group, so that there is a unique functionality, which may be differentially functionalized. By using protective groups, one can distinguish a side-chain functionality from a terminal amino acid functionality. Also, by appropriate design, one may provide for preferential reaction between the same functionalities present at different sites on the linking group. Whether one uses synthesis or cloning for preparation of oligopeptides, will to a substantial degree depend on the length of the linker.

### Methods of Using Binding Compositions of the Invention

In one aspect, the invention provides a method for detecting or measuring one or more target analytes from biological sources. Conventional methodologies are employed to prepare samples for analysis. Preparative techniques include mild cell lysis by osmotic disruption of cellular membranes, to enzymatic digestion of connective tissue followed by osmotic-based lysis, to mechanical homogenization, to ultrasonication.

For sources containing target polynucleotides, guidance for sample preparation techniques can be found in standard treatises, such as Sambrook et al, Molecular Cloning, Second Edition (Cold Spring Harbor Laboratory Press, New York, 1989); Innis et al, editors, PCR Protocols (Academic Press, New York, 1990); Berger and Kimmel, “Guide to Molecular Cloning Techniques ,” Vol. 152, Methods in Enzymology (Academic Press, New York, 1987); or the like. For mammalian tissue culture cells, or like sources, samples of target RNA may be prepared by conventional cell lysis techniques (e.g. 0.14 M NaCl, 1.5 mM MgCl2, 10 mM Tris-Cl (pH 8.6), 0.5% Nonidet P-40, 1 mM dithiothreitol, 1000 units/mL placential RNAase inhibitor or 20 mM vanadyl-ribonucleoside complexes).

In carrying out the assays, the components, i.e., the sample, composition of microparticles, and in some embodiments a cleavage-inducing moiety, are combined in an assay medium in any order, usually simultaneously. Alternatively, one or more of the reagents may be combined with one or more of the remaining agents to form a subcombination. The subcombination can then be subjected to incubation. Then, the remaining reagents or subcombination thereof may be combined and the mixture incubated. The amounts of the reagents are usually determined empirically. The components are combined under binding conditions, usually in an aqueous medium, generally at a pH in the range of about 5 to about 10, with buffer at a concentration in the range of about 10 to about 200 mM. These conditions are conventional, where conventional buffers may be used, such as phosphate, carbonate, HEPES, MOPS, Tris, borate, etc., as well as other conventional additives, such as salts, stabilizers, organic solvents, etc. The aqueous medium may be solely water or may include from 0.01 to 80 or more volume percent of a co-solvent.

The combined reagents are incubated for a time and at a temperature that permit a substantial number of binding events to occur. The time for incubation after combination of the reagents varies depending on the (i) nature and expected concentration of the analyte being detected, (ii) the mechanism by which the binding compounds for complexes with analytes, and (iii) the affinities of the specific reagents employed. Moderate temperatures are normally employed for the incubation and usually constant temperature. Incubation temperatures will normally range from about 5° to 99° C., usually from about 15° to 85° C., more usually 35° to 75° C.

For quantitation, one may choose to use controls, which provide a signal in relation to the amount of the target that is present or is introduced. A control to allow conversion of relative fluorescent signals into absolute quantities is accomplished by addition of a known quantity of a fluorophore to each sample before separation of the molecular tags. Any fluorophore that does not interfere with detection of the molecular tag signals can be used for normalizing the fluorescent signal. Such standards preferably have separation properties that are different from those of any of the molecular tags in the sample, and could have the same or a different emission wavelength. Exemplary fluorescent molecules for standards include ROX, FAM, and fluorescein and derivatives thereof.

### EXAMPLES

The invention is demonstrated further by the following syntheses and illustrative examples. Parts and percentages are by weight unless otherwise indicated. Temperatures are in degrees Centigrade (° C.) unless otherwise specified. The following preparations and examples illustrate the invention but are not intended to limit its scope. Unless otherwise indicated, peptides used in the following examples were prepared by synthesis using an automated synthesizer and were purified by gel electrophoresis or HPLC.

The following abbreviations have the meanings set forth below:

Tris HCl—Tris(hydroxymethyl)aminomethane-HCl (a 10× solution) from BioWhittaker, Walkersville, Md.

TLC—thin layer chromatography

BSA—bovine serum albumin, e.g. available from Sigma Chemical Company (St. Louis, Mo.), or like reagent supplier.

EDTA—ethylene diamine tetra-acetate from Sigma Chemical Company

FAM—carboxyfluorescein

EMCS—N-ε-maleimidocaproyloxy-succinimide ester

EDC—1-Ethyl-3-(3-dimethylaminopropyl)carbodiimide

NHS—N-hydroxysuccinimide

DCC—1,3-dicylcohexylcarbodiimide

DMF—dimethylformamide

Fmoc—N-(9-fluorenylmethoxycarbonyl)-

### Example 1

### Preparation of Aminodextran Derivatized Microspheres

Aminodextran is prepared as described in Pollner, U.S. Pat. No. 6,346,384. Briefly, hydroxypropylaminodextran (1NH₂/16 glucose) is prepared by dissolving Dextran T-500 (Pharmacia, Uppsala, Sweden) (100 g) in 500 mL of H₂O in a 3-neck round-bottom flask equipped with mechanical stirrer and dropping funnel. To the above solution is added 45 g sodium hydroxide, 50 mg EDTA, 50 mg NaBH₄, 50 mg hydroquinone and 200 g N-(2,3-epoxypropyl) phthalimide. The mixture is heated and stirred in a 90° C. water bath for 2 hr. A small aliquot is precipitated three times from methanol and analyzed by NMR. Appearance of a peak at 7.3-7.66 indicates incorporation of phthalimide. The main reaction mixture is precipitated by addition to 3.5 L of methanol and the solid is collected. The phthalimide protecting group is removed by dissolving the product above in 500 mL of 0.1 M acetate buffer, adding 50 mL of 35% hydrazine and adjusting the pH to 3.5. The mixture is heated at 80° C. for 1 hr, the pH is readjusted to 3.2, and the mixture is heated for an additional one-half hour. An aliquot is precipitated three times in methanol. The reaction mixture is neutralized to pH 8 and stored at room temperature. The product is purified by tangential flow filtration using a 50,000 molecular weight cut-off filter, washing with about 8 L water, 0.5 L of 0.1M HCl, 0.5 L of 0.01 M NaOH, and finally 3 L of water. The product solution is concentrated by filtration to 700 mL and then is lyophilized. Determination of reactive amines using trinitrobenzenesulfonate indicates about 1 amine per 16 glucose residues.

A solution of hydroxypropylaminodextran (synthesized as described above) is prepared at 2 mg/mL in 50 mM MES (pH 6). One hundred fifty (150) mg carboxyl-modified microspheres (Bangs Laboratories, Fishers, Ind.) in 7.5 mL water is added dropwise to 7.5 mL of the hydroxypropylaminodextran solution while vortexing. One hundred eighty eight (188) μL of EDAC solution (80 mg/mL) in water is added to the coating mixture while vortexing. The mixture is incubated overnight at room temperature in the dark. The mixture is diluted with 12 mL water and centrifuged. The supernatant is discarded and the bead pellet is suspended in 40 mL water by sonication. The beads are washed 3 times with water (40 mL per wash) by repeated centrifugation and suspension by sonication. The final pellet is suspended in 5 mL water.

### Example 2

### Conjugation and Release of a Molecular Tag

FIGS. 7A-B summarize the methodology for conjugation of molecular tag precursor to an antibody or other binding moiety with a free amino group, and the reaction of the resulting conjugate with singlet oxygen to produce a sulfinic acid moiety as the released molecular tag. FIGS. 8A-J shows several molecular tag reagents, most of which utilize 5- or 6-carboxyfluorescein (FAM) as starting material.

### Example 3

### Preparation of Pro2, Pro4, and Pro6 through Pro13

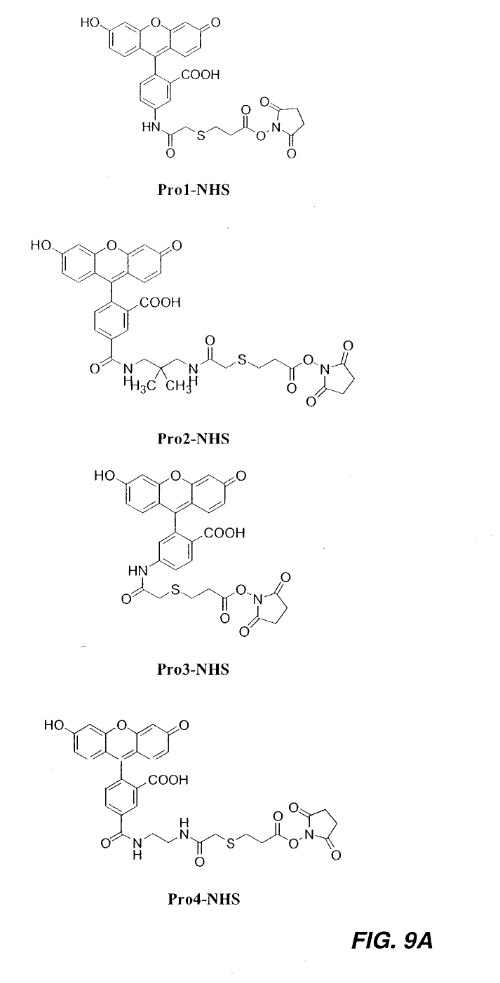

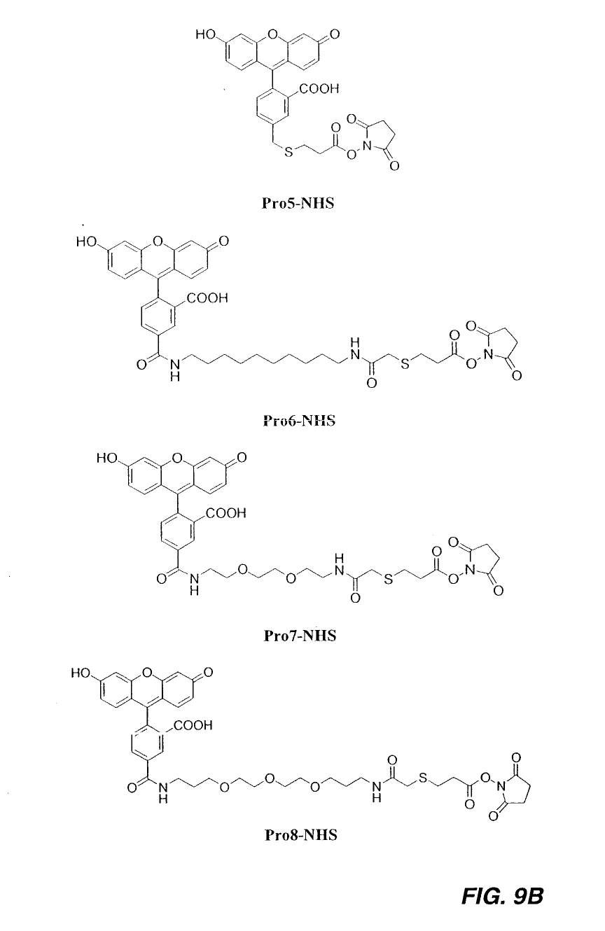

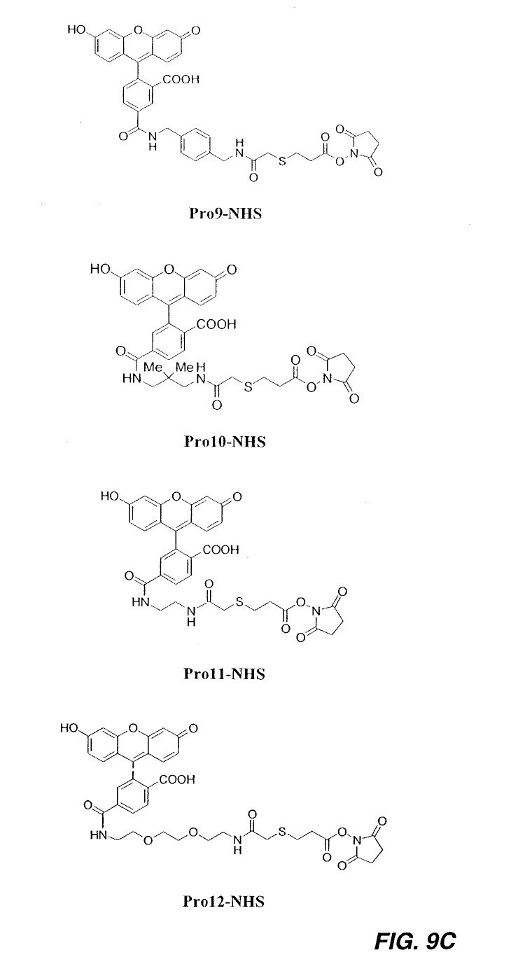

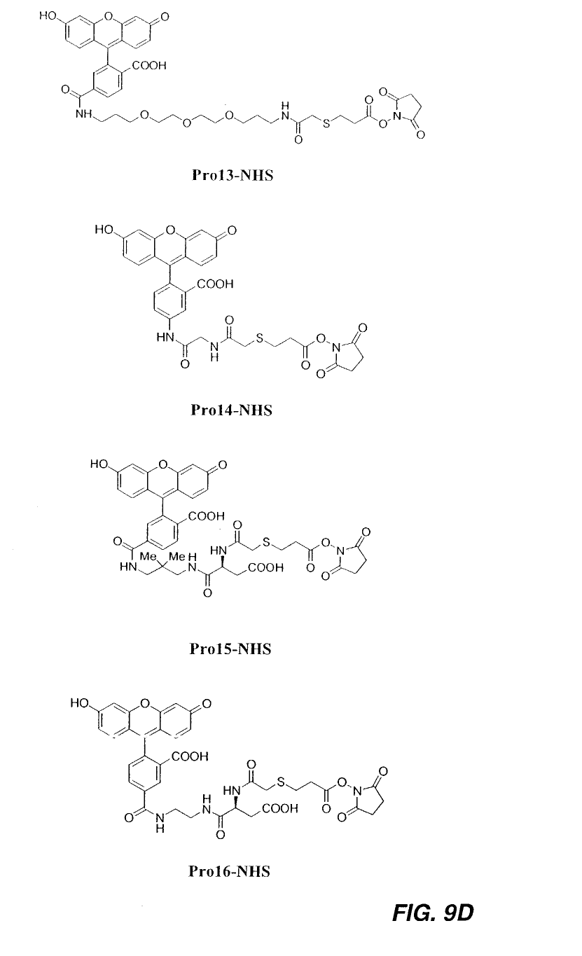

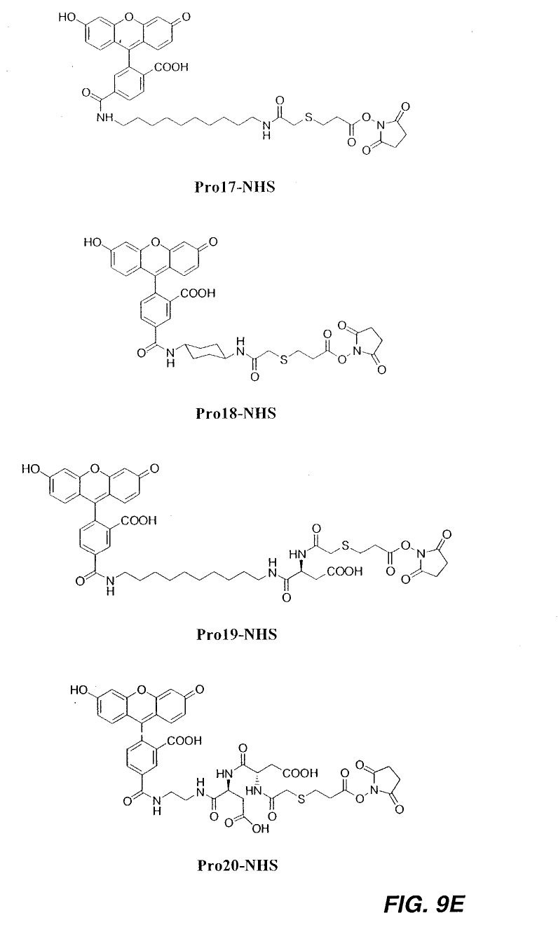

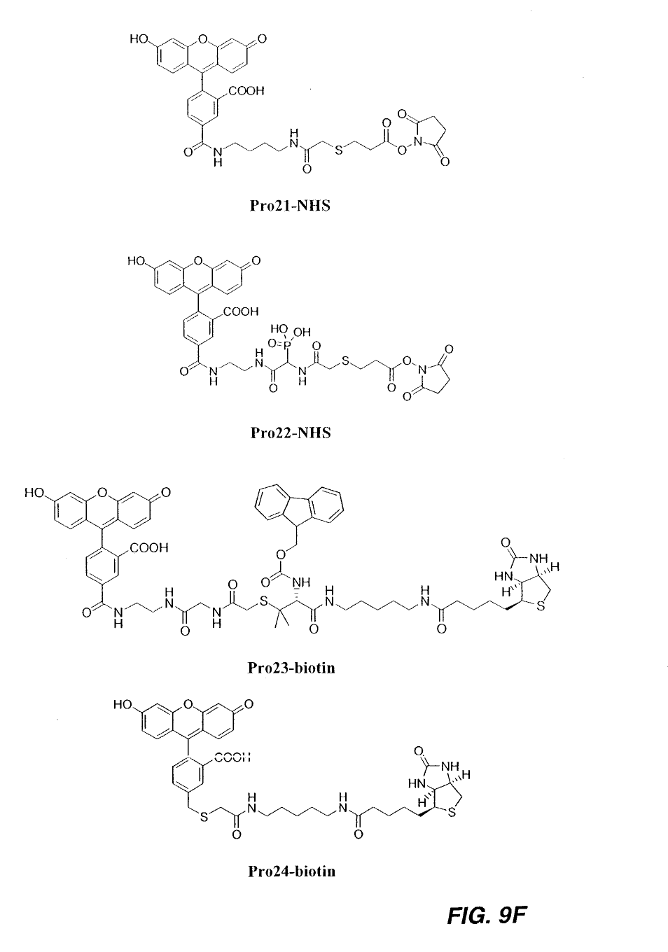

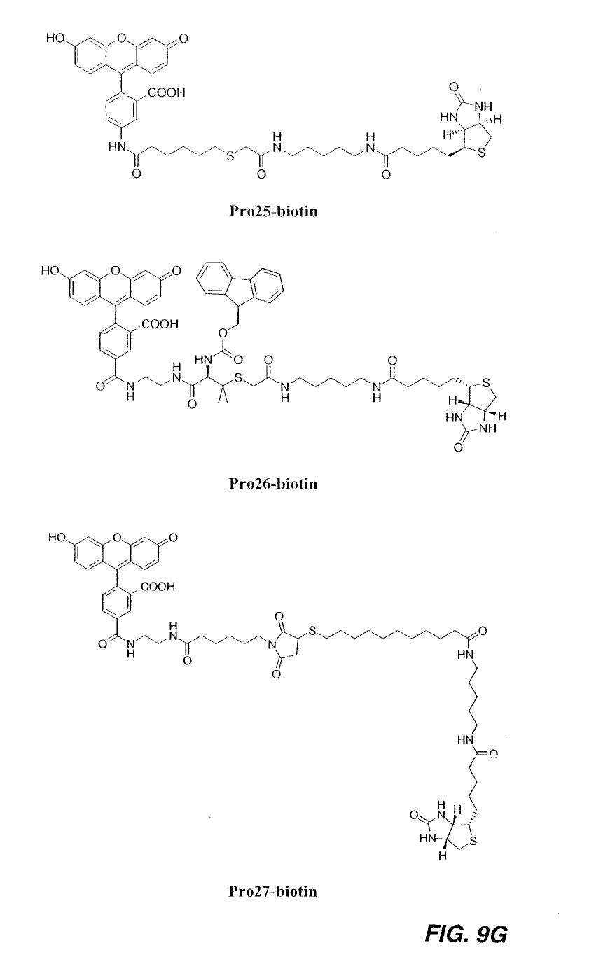

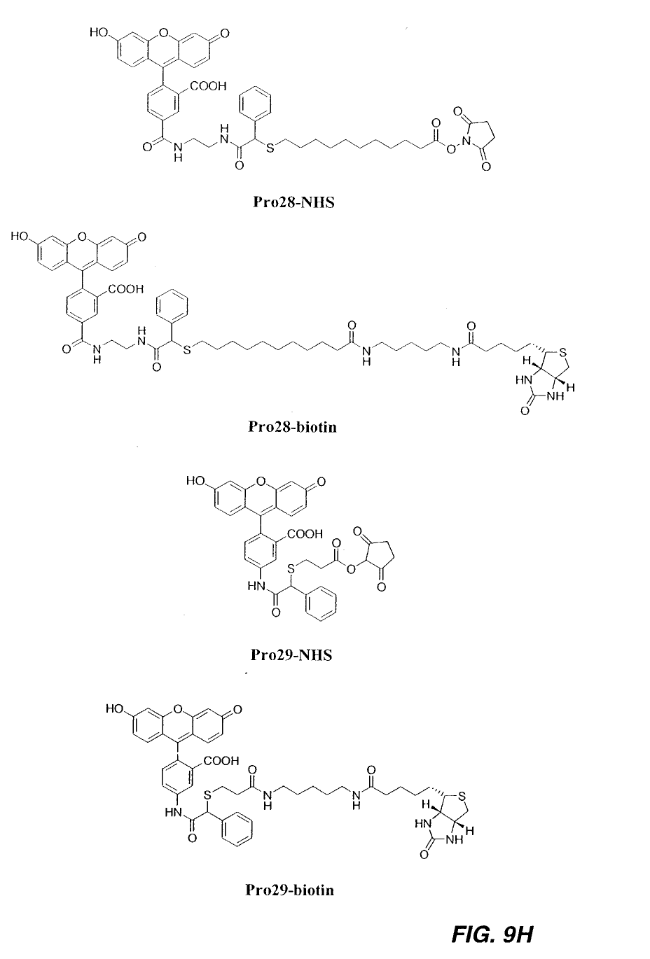

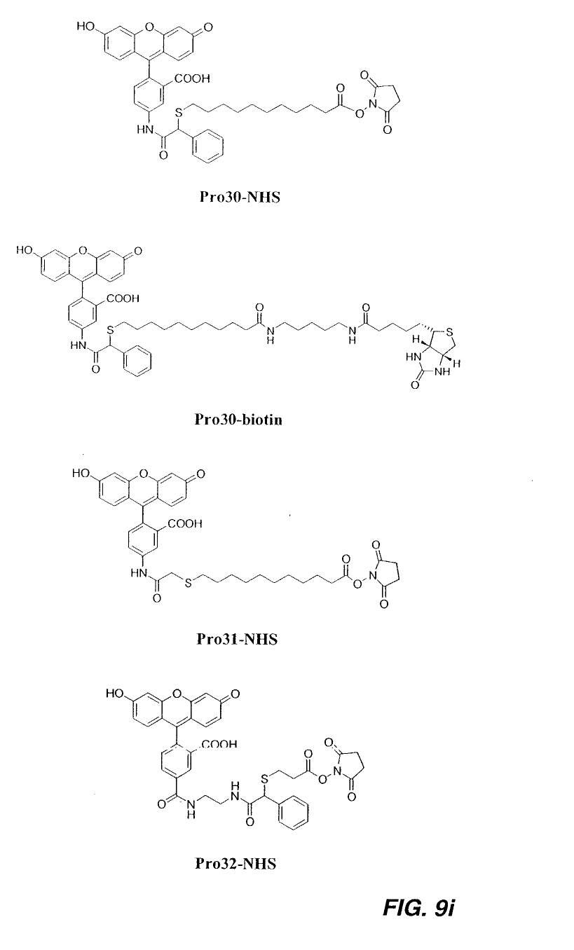

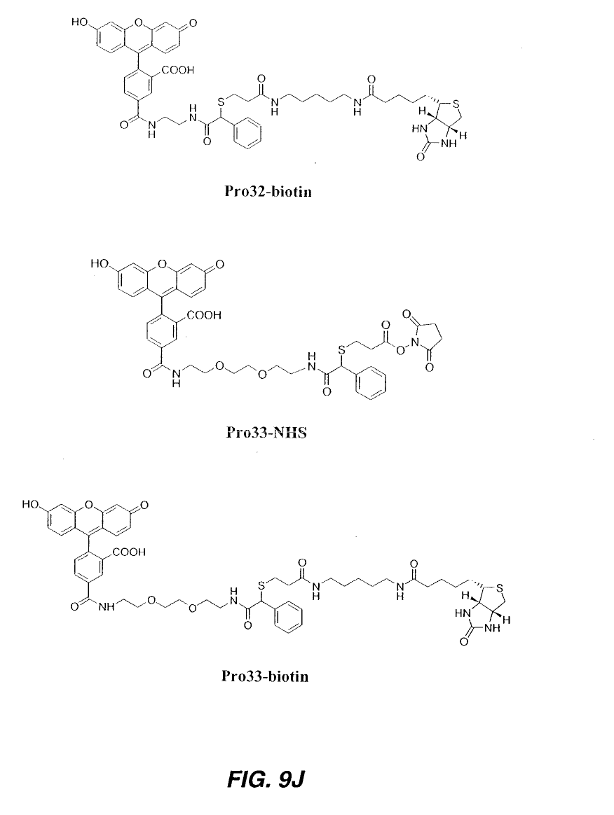

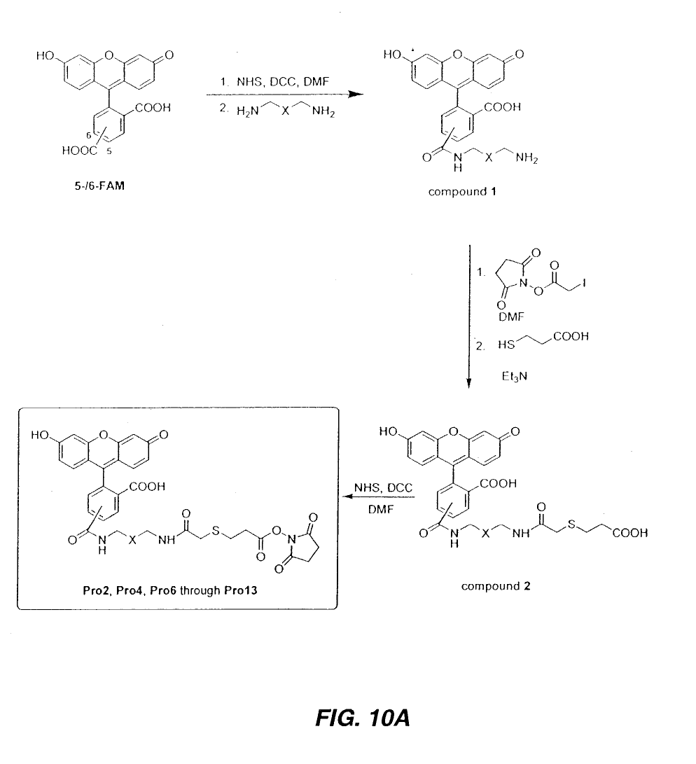

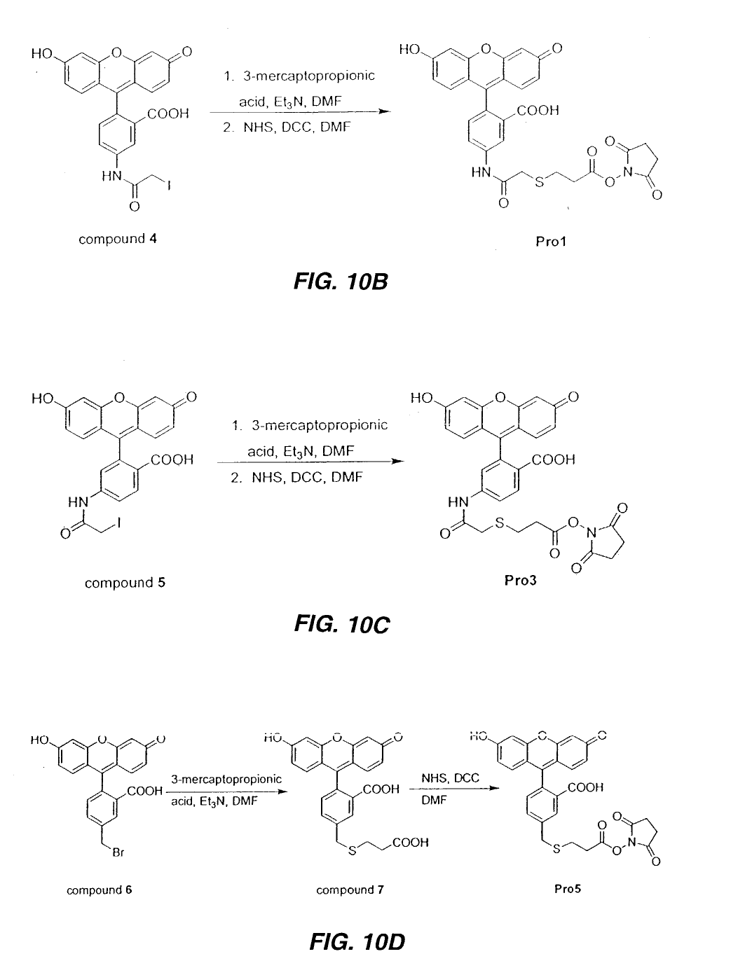

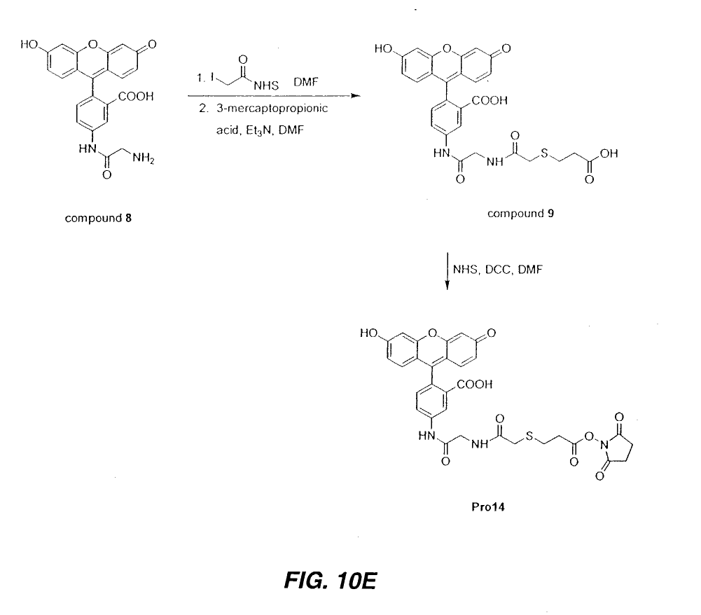

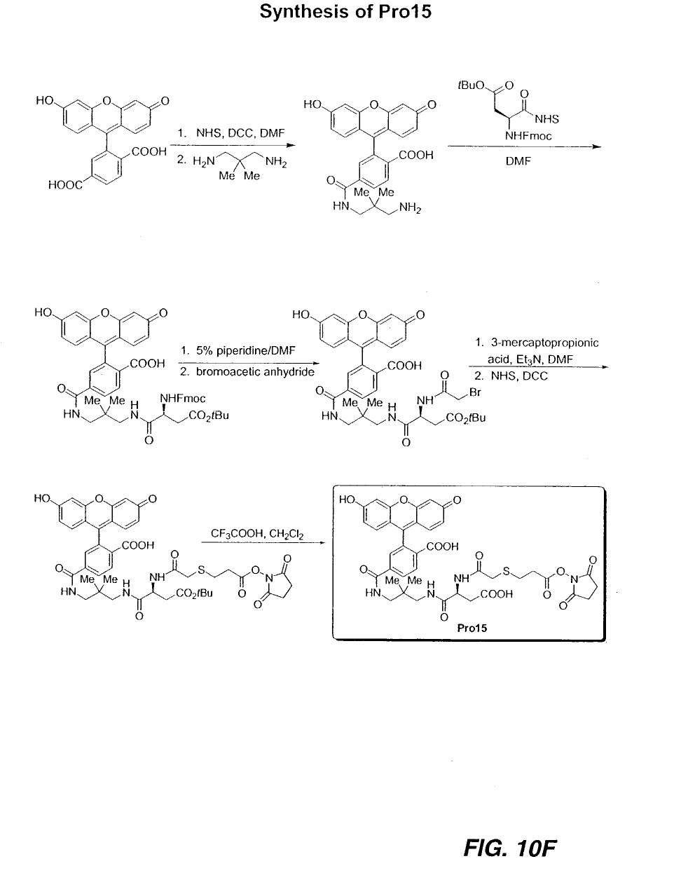

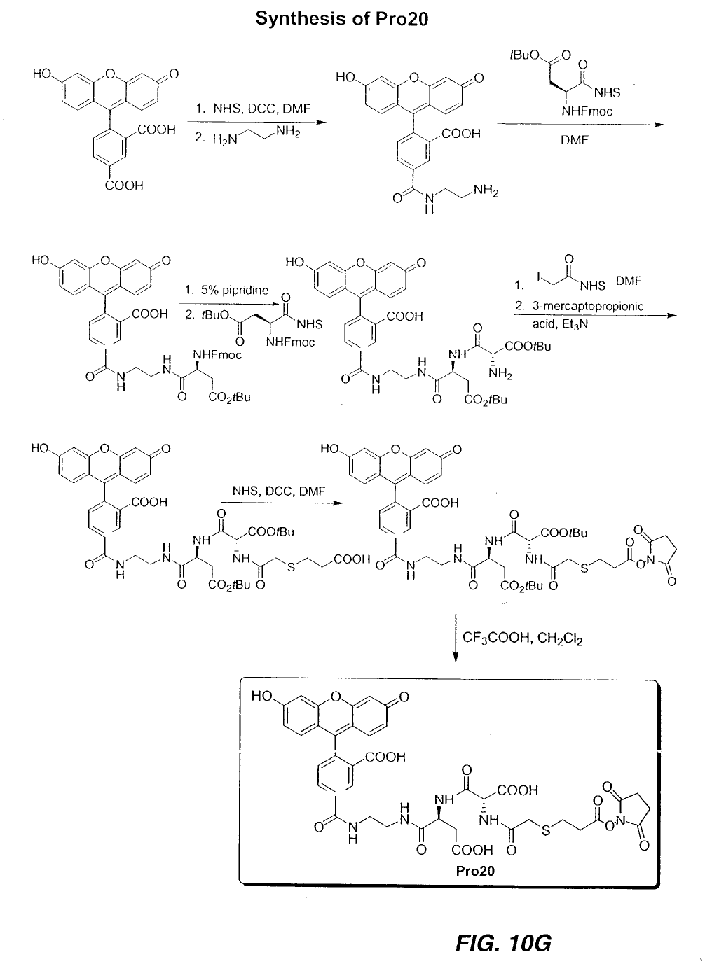

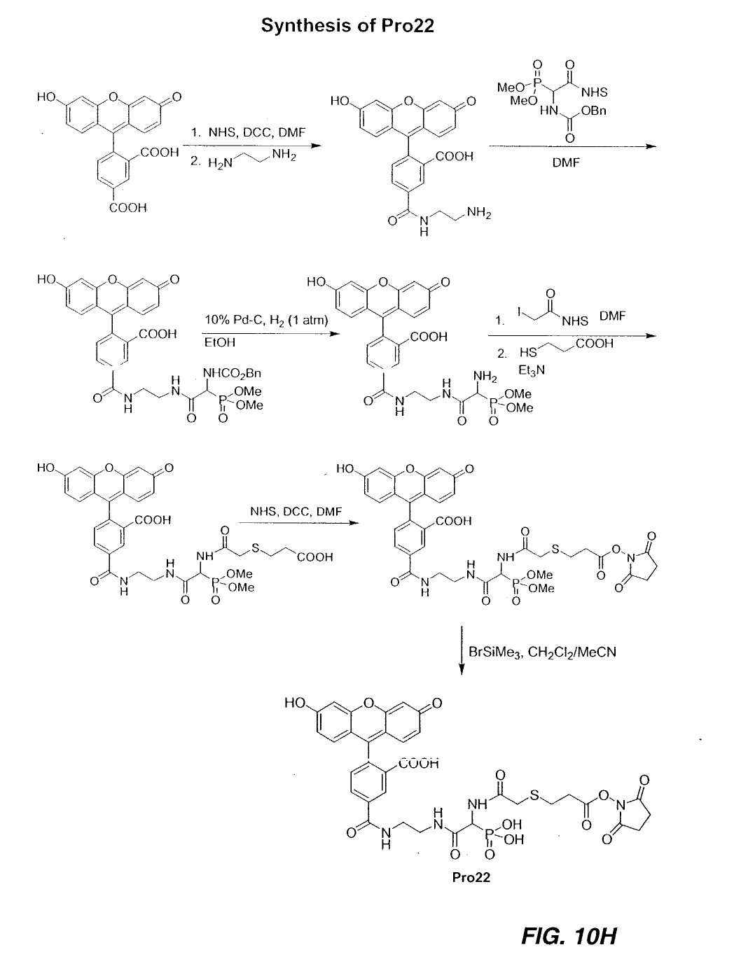

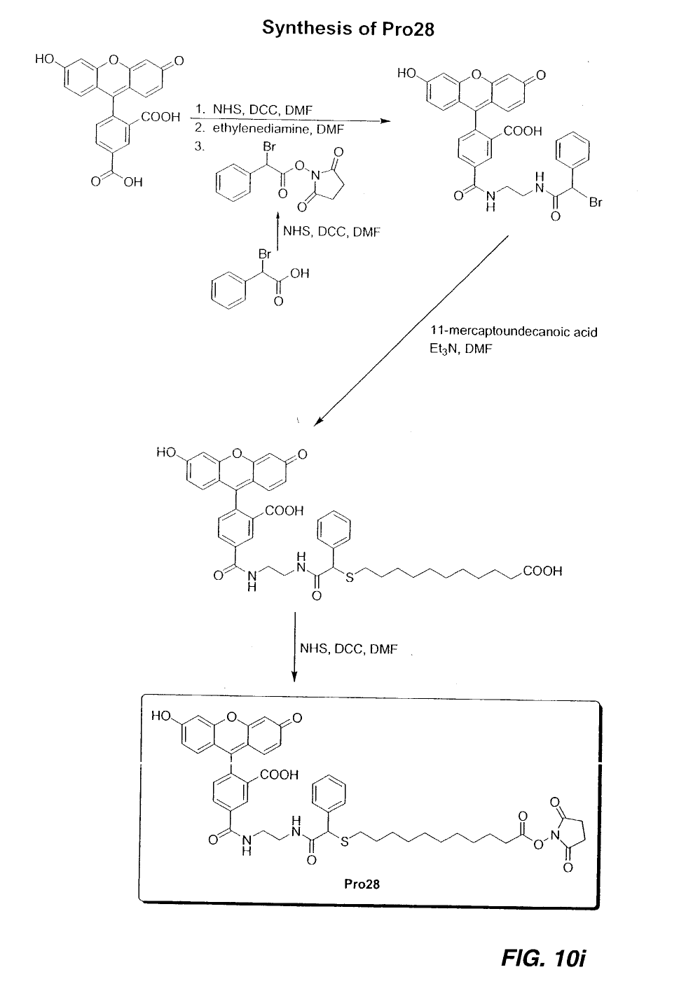

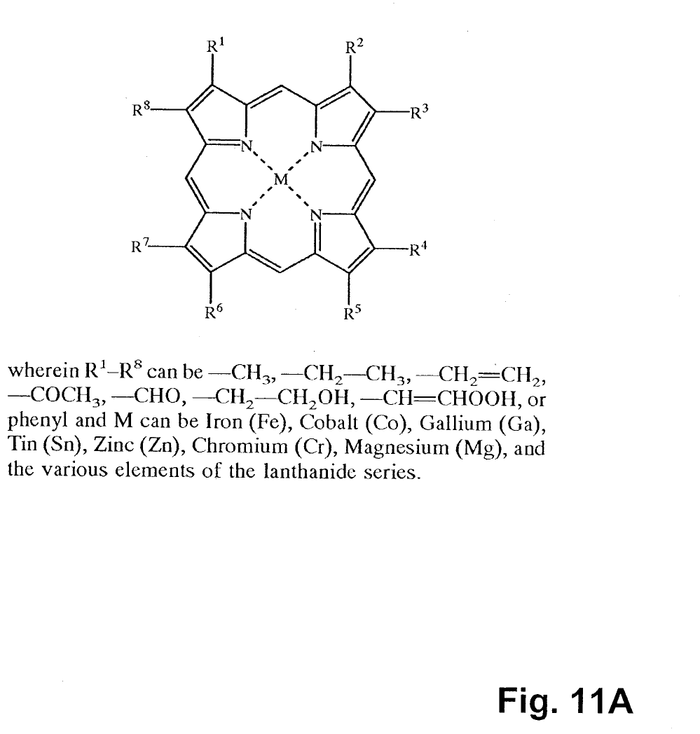

The scheme outlined in FIG. 9A shows a five-step procedure for the preparation of the carboxyfluorescein-derived molecular tag precursors, namely, Pro2, Pro4, Pro6, Pro7, Pro8, Pro9, Pro10, Pro11, Pro12, and Pro13. The first step involves the reaction of a 5- or 6-FAM with N-hydroxysuccinimide (NHS) and 1,3-dicylcohexylcarbodiimide (DCC) in DMF to give the corresponding ester, which was then treated with a variety of diamines to yield the desired amide, compound 1. Treatment of compound 1 with N-succinimidyl iodoacetate provided the expected iodoacetamide derivative, which was not isolated but was further reacted with 3-mercaptopropionic acid in the presence of triethylamine. Finally, the resulting β-thioacid (compound 2) was converted, as described above, to its NHS ester. The various e-tag moieties were synthesized starting with 5- or 6-FAM, and one of various diamines. The diamine is given H₂N\hat{ }X\hat{ }NH₂ in the first reaction of FIG. 9A. The regioisomer of FAM and the chemical entity of “X” within the diamine are indicated in the table below for each of the molecular tag precursors synthesized. Clearly, the diamine, X, can have a wide range of additional forms, as described above in the discussion of the mobility modifier moiety.

| Precursor | FAM | X |
|---|---|---|
| Pro2 | 5-FAM | C(CH₃)₂ |
| Pro4 | 5-FAM | no carbon |
| Pro6 | 5-FAM | (CH₂)₈ |
| Pro7 | 5-FAM | CH₂OCH₂CH₂OCH₂ |
| Pro8 | 5-FAM | CH₂CH₂OCH₂CH₂OCH₂CH₂OCH₂CH₂ |
| Pro9 | 5-FAM | 1,4-phenyl |
| Pro10 | 6-FAM | C(CH₃)₂ |
| Pro11 | 6-FAM | no carbon |
| Pro12 | 6-FAM | CH₂OCH₂CH₂OCH² |
| Pro13 | 6-FAM | CH₂CH₂OCH₂CH₂OCH₂CH₂OCH₂CH₂ |

Synthesis of Compound 1

To a stirred solution of 5- or 6-carboxyfluorescein (0.5 mmol) in dry DMF (5 mL) were added N-hydroxysuccinimide (1.1 equiv.) and 1,3-dicylcohexylcarbodiimide (1.1 equiv.). After about 10 minutes, a white solid (dicyclohexylurea) started forming. The reaction mixture was stirred under nitrogen at room temperature overnight. TLC (9:1 CH₂Cl₂—MeOH) indicated complete disappearance of the starting material.

The supernatant from the above mixture was added dropwise to a stirred solution of diamine (2-5 equiv.) in DMF (10 mL). As evident from TLC (40:9:1 CH₂Cl₂—MeOH—H₂O), the reaction was complete instantaneously. The solvent was removed under reduced pressure. Flash chromatography of the resulting residue on latrobeads silica provided the desired amine (compound 1) in 58-89% yield. The ¹H NMR (300 MHz, DMSO-d₆) of compound 1 was in agreement with the assigned structure.

Synthesis of Compound 2

To the amine (compound 1) (0.3 mmol) were sequentially added dry DMF (10 mL) and N-succinimidyl iodoacetate (1.1 equiv.). The resulting mixture was stirred at room temperature until a clear solution was obtained. TLC (40:9:1 CH₂Cl₂—MeOH—H₂O) revealed completion of the reaction.

The above reaction solution was then treated with triethylamine (1.2 equiv.) and 3-mercaptopropionic acid (3.2 equiv.). The mixture was stirred at room temperature overnight. Removal of the solvent under reduced pressure followed by flash chromatography afforded the β-thioacid (compound 2) in 62-91% yield. The structure of compound 2 was assigned on the basis of its ¹NMR (300 MHz, DMSO-d₆).

Synthesis of Pro2, Pro4, and Pro6 through Pro13

To a stirred solution of the β-thioacid (compound 2) (0.05 mmol) in dry DMF (2 mL) were added N-hydroxysuccinimide (1.5 equiv.) and 1,3-dicylcohexylcarbodiimide (1.5 equiv.). The mixture was stirred at room temperature under nitrogen for 24-48 h (until all of the starting material had reacted). The reaction mixture was concentrated under reduced pressure and then purified by flash chromatography to give the target molecule in 41-92% yield.

Preparation of Pro1

The compounds of this reaction are shown in FIG. 9B. To a stirred solution of 5-iodoacetamidofluorescein (compound 4) (24 mg, 0.047 mmol) in dry DMF (2 mL) were added triethylamine (8 μL, 0.057 mmol) and 3-mercaptopropionic acid (5 μL, 0.057 mmol). The resulting solution was stirred at room temperature for 1.5 h. TLC (40:9:1 CH₂Cl₂—MeOH—H₂O) indicated completion of the reaction. Subsequently, N-hydroxysuccinimide (9 mg, 0.078 mmol) and 1,3-dicylcohexylcarbodiimide (18 mg, 0.087 mmol) were added. The reaction mixture was stirred at room temperature under nitrogen for 19 h at which time TLC showed complete disappearance of the starting material. Removal of the solvent under reduced pressure and subsequent flash chromatography using 25:1 and 15:1 CH₂Cl₂—MeOH as eluant afforded Pro1 (23 mg, 83%).

Preparation of Pro3

The compounds of this reaction are shown in FIG. 9C. To a stirred solution of 6-iodoacetamidofluorescein (compound 5) (26 mg, 0.050 mmol) in dry DMF (2 mL) were added triethylamine (8 μL, 0.057 mmol) and 3-mercaptopropionic acid (5 μL, 0.057 mmol). The resulting solution was stirred at room temperature for 1.5 h. TLC (40:9:1 CH₂Cl₂—MeOH—H₂O) indicated completion of the reaction. Subsequently, N-hydroxysuccinimide (11 mg, 0.096 mmol) and 1,3-dicylcohexylcarbodiimide (18 mg, 0.087 mmol) were added. The reaction mixture was stirred at room temperature under nitrogen for 19 h at which time TLC showed complete disappearance of the starting material. Removal of the solvent under reduced pressure and subsequent flash chromatography using 30:1 and 20:1 CH₂Cl₂—MeOH as eluant provided Pro3 (18 mg, 61%).

Preparation of Pro5

The compounds of this reaction are shown in FIG. 9D.

Synthesis of Compound 7

To a stirred solution of 5-(bromomethyl)fluorescein (compound 6) (40 mg, 0.095 mmol) in dry DMF (5 mL) were added triethylamine (15 μL, 0.108 mmol) and 3-mercaptopropionic acid (10 μL, 0.115 mmol). The resulting solution was stirred at room temperature for 2 days. TLC (40:9:1 CH₂Cl₂—MeOH—H₂O) indicated completion of the reaction. The reaction solution was evaporated under reduced pressure. Finally, flash chromatography employing 30:1 and 25:1 CH₂Cl₂—MeOH as eluant provided the β-thioacid (compound 7) (28 mg, 66%).

Synthesis of Pro5

To a solution of the acid (compound 7) (27 mg, 0.060 mmol) in dry DMF (2 mL) were added N-hydroxysuccinimide (11 mg, 0.096 mmol) and 1,3-dicylcohexylcarbodiimide (20 mg, 0.097 mmol). The reaction mixture was stirred at room temperature under nitrogen for 2 days at which time TLC (9:1 CH₂Cl₂—MeOH) showed complete disappearance of the starting material. Removal of the solvent under reduced pressure and subsequent flash chromatography with 30:1 CH₂Cl₂—MeOH afforded Pro5 (24 mg, 73%).

Preparation of Pro14

The compounds of this reaction are shown in FIG. 9E.

Synthesis of Compound 9

To 5-aminoacetamidofluorescein (compound 8) (49 mg, 0.121 mmol) were sequentially added dry DMF (4 mL) and N-succinimidyl iodoacetate (52 mg, 0.184). A clear solution resulted and TLC (40:9:1 CH₂Cl₂—MeOH—H20) indicated complete disappearance of the starting material.

The above reaction solution was then treated with triethylamine (30 μL, 0.215 mmol) and 3-mercaptopropionic acid (30 μL, 0.344 mmol). The resulting mixture was stirred for 2 h. Removal of the solvent under reduced pressure followed by flash chromatography using 20:1 and 15:1 CH₂Cl₂—MeOH as eluant gave the β-thioacid (compound 9) (41 mg, 62%). The structural assignment was made on the basis of ¹NMR (300 MHz, DMSO-d₆).

Synthesis of Pro14

To a stirred solution of compound 9 (22 mg, 0.04 mmol) in dry DMF (2 mL) were added N-hydroxysuccinimide (9 mg, 0.078 mmol) and 1,3-dicylcohexylcarbodiimide (16 mg, 0.078 mmol). The resulting solution was stirred at room temperature under nitrogen for about 24 h. The reaction mixture was concentrated under reduced pressure and the residue purified by flash chromatography using 30:1 and 20:1 CH₂Cl₂—MeOH as eluant to give Pro14 (18 mg, 70%).

Synthesis of Pro15, Pro20, Pro22, and Pro28

The synthesis schemes for producing NHS esters of molecular tags Pro15, Pro20, Pro22, and Pro28 are shown in FIGS. 16F-I, respectively. All of the reagent and reaction conditions are conventional in the art and proceed similarly as the reactions described above.

What is claimed is:

1. A method of generating molecular tags indicative of a plurality of polynucleotides in a sample, the method comprising the steps of:
extending a primer annealed to each polynucleotide to form a detection probe under conditions that permit dissociation of detection probes from the polynucleotides after extension, each detection probe having a molecular tag and either a sensitizer with an effective proximity or a capture moiety, the molecular tag being attached by a cleavable linkage and being within the effective proximity of the sensitizer upon dissociation of the detection probe from the polynucleotide whenever the detection probe has a sensitizer attached, and the molecular tag being selected from a plurality of molecular tags such that each molecular tag of the plurality has one or more physical and/or optical characteristics distinct from those of the other molecular tags of the plurality so that each molecular tag forms a distinguishable peak upon cleavage and separation based on such one or more physical and/or optical characteristics;
generating detectable amounts of detection probes in said step of extending;
activating the sensitizers to generate an active species so that the cleavable linkages are cleaved and the molecular tags are released; and
separating and identifying the released molecular tags to determine the plurality of polynucleotides in the sample.

2. The method of claim 1 wherein said step of extending includes extending with a DNA polymerase said primer by a terminator, the terminator having said sensitizer attached or said capture moiety attached.

3. The method of claim 2 wherein said terminator has said capture moiety attached and wherein after said step of generating detectable amounts of said detection probes, a further step of capturing each of said detection probes by a complementary moiety of said capture moiety, the complementary moiety being attached to a photosensitizer bead.

4. The method of claim 3 wherein said capture moiety is biotin and said complementary moiety is avidin or streptavidin.

5. The method according to claims 1, 2, 3, or 4 wherein said step of separating is electrophoretically separating or chromatographically separating, and wherein said molecular tag has a molecular weight in the range of from 100 to 2500 daltons.

6. The method of claim 5 wherein said molecular tags consist of said plurality of molecular tags selected from a group defined by the formula:
—L—(M,D)
wherein:
L is a cleavable linkage;
D is a detection moiety; and
M is a bond or a water soluble organic compound consisting of from 1 to 100 atoms, not including hydrogen, that are selected from the group consisting of carbon, oxygen, nitrogen, phosphorus, boron, and sulfur.

7. The method of claim 6 wherein said plurality is in the range of from 2 to 100 and wherein D is a fluorescent label.

8. A composition of matter defined by the formula: 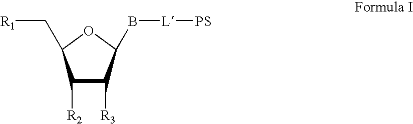
wherein:
B is a nucleobase;
R₁ is —OH, or mono-, di-, or triphosphate, or an analog thereof;
R₂ is —OH, H, F, Cl, NH₂, N₃, or OR′ where R′ is C1-C6 alkyl;
R₃ is —OH, H, F, Cl, NH₂, N₃, or OR′.
L′ is a diradical moiety of from 1 to 50 atoms selected from the group consisting of hydrogen, carbon, oxygen, nitrogen, phosphorus, and sulfur.
PS is a photosensitizer.

9. A composition of matter defined by the formula: 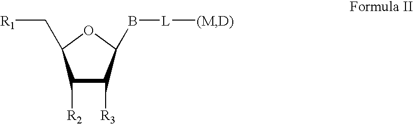
wherein:
B is a nucleobase;
R₁ is —OH, or mono-, di-, or triphosphate, or an analog thereof;
R₂ is —OH, H, F, Cl, NH₂, N₃, or OR′ where R′ is C1-C6 alkyl;
R₃ is —OH, H, F, Cl, NH₂, N₃, or OR′.
L is a cleavable linkage;
D is a detection moiety;
M is a bond or a water soluble organic compound consisting of from 1 to 100 atoms, not including hydrogen, that are selected from the group consisting of carbon, oxygen, nitrogen, phosphorus, boron, and sulfur.

10. The composition of claim 9 wherein L is selected from the group consisting of olefins, thioethers, selenoethers, thiazoles, oxazoles, and imidazoles having from 6 to 100 atoms, not including hydrogen, selected from the group consisting of carbon, oxygen, nitrogen, phosphorus, boron, and sulfur.

11. A composition of matter comprising:
one or more photosensitizer beads having a complementary moiety attached, the complementary moiety being capable of capturing a capture moiety; and
one or more oligonucleotides each having attached a capture moiety and a molecular tag, the molecular tag being attached by a cleavable linkage, and each molecular tag being selected from a plurality of molecular tags such that each molecular tag of the plurality has one or more physical and/or optical characteristics distinct from those of the other molecular tags of the plurality so that each molecular tag forms a distinguishable peak upon cleavage and separation based on such one or more physical and/or optical characteristics;
wherein each of the one or more oligonucleotides are attached to the one or more photosensitizer beads by specific binding of the capture moiety to the complementary moiety.

12. The composition of claim 11 wherein said separation is electrophoretic separation or chromatographic separation, and wherein said molecular tag has a molecular weight in the range of from 100 to 2500 daltons.

13. The composition of claim 12 wherein each of said molecular tags attached to said one or more oligonucleotides is selected from a group defined by the formula:
—L—(M,D)
wherein:
L is a cleavable linkage;
D is a detection moiety; and
M is a bond or a water soluble organic compound consisting of from 1 to 100 atoms, not including hydrogen, that are selected from the group consisting of carbon, oxygen, nitrogen, phosphorus, boron, and sulfur.

14. The composition of claim 13 wherein D is a fluorescent label, a chromogenic label, or an electrochemical label.

15. The composition of claim 14 wherein M is a polymer selected from any one of polyethers, polyesters, polypeptides, oligosaccharides, polyurethanes, polyamides, polysulfonamides, polysulfoxides, polyphosphonates, and block copolymers thereof.

16. The composition of claim 15 wherein D is a fluorescein.

17. The composition of claim 16 wherein said fluorescein is selected from the group consisting of 5- and 6-carboxyfluorescein, 5- and 6-carboxy-4,7-dichlorofluorescein, 2′,7′-dimethoxy-5- and 6-carboxy-4,7-dichlorofluorescein, 2′,7′-dimethoxy-4′,5′-dichloro-5- and 6-carboxyfluorescein, 2′,7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy-4,7-dichlorofluorescein, 2′,7′-dichloro-5- and 6-carboxy-4,7-dichlorofluorescein, and 2′,4′,5′,7′-tetrachloro-5- and 6-carboxy-4,7-dichlorofluorescein.

18. The composition of claim 13 wherein L is selected from the group consisting of olefins, thioethers, selenoethers, thiazoles, oxazoles, and imidazoles.

19. The composition in accordance with claims 11, 12, 13, 14, 15, 16, 17, or 18 wherein said plurality of molecular tags is in the range of from 2 to 100, and wherein said separation is electrophoretic separation.

20. The composition of claim 19 wherein said plurality of molecular tags is in the range of from 3 to 50.


SEQUENCE LISTING (1 sequence)

SEQ ID NO: 1 - DNA, 44 bp, Artificial Sequence
  No special biological significance.
accaccaccctaatacgactcactatagggaccataccaaccag